SEQUENCE LISTING (74 sequences)

SEQ ID NO: 1 - protein, 1275 aa, synthetic construct
  REGION 1..1275: note = HER2
MELAALCRWGLLLALLPPGAASGGSRLEEELRRRLTEGGGSGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 2 - protein, 615 aa, synthetic construct
  REGION 1..615: note = CD30
MRVLLAALGLLFLGALRAGGSRLEEELRRRLTEGGGSGFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 3 - protein, 249 aa, synthetic construct
  REGION 1..249: note = CD79B
MARLALSPVPSHWMVALLLLLSAEPVPAGGSRLEEELRRRLTEGGGSGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 4 - protein, 530 aa, synthetic construct
  REGION 1..530: note = Nectin-4
MPLSLGAEMWGPEAWLLLLLLLASFTGRCPAGGSRLEEELRRRLTEGGGSGGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 5 - protein, 203 aa, synthetic construct
  REGION 1..203: note = BCMA
MGSRLEEELRRRLTEGGGSGLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 6 - protein, 1230 aa, synthetic construct
  REGION 1..1230: note = EGFR
MRPSGTAGAALLALLAALCPASRAGGSRLEEELRRRLTEGGGSGLEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 7 - protein, 384 aa, synthetic construct
  REGION 1..384: note = CD33
MPLLLLLPLLWAGALAMGGSRLEEELRRRLTEGGGSGDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 8 - protein, 316 aa, synthetic construct
  REGION 1..316: note = CD20
MGSRLEEELRRRLTEGGGSGTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 9 - protein, 867 aa, synthetic construct
  REGION 1..867: note = CD22
MHLLGPWLLLLVLEYLAFSGGSRLEEELRRRLTEGGGSGDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 10 - protein, 576 aa, synthetic construct
  REGION 1..576: note = CD19
MPPPRLLFFLLFLTPMEVRGGSRLEEELRRRLTEGGGSGPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTR

SEQ ID NO: 11 - protein, 343 aa, synthetic construct
  REGION 1..343: note = TROP2
MARGPGLAPPPLRLPLLLLVLAAVTGGGSRLEEELRRRLTEGGGSGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 12 - protein, 554 aa, synthetic construct
  REGION 1..554: note = B7-H3
MLRRRGSPGMGVHVGAALGALWFCLTGAGGSRLEEELRRRLTEGGGSGLEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 13 - protein, 254 aa, synthetic construct
  REGION 1..254: note = FOLR1
MAQRMTTQLLLLLVWVAVVGEAQTGGSRLEEELRRRLTEGGGSGRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMS

SEQ ID NO: 14 - protein, 1326 aa, synthetic construct
  REGION 1..1326: note = CD45
MTMYLWLKLLAFGFAFLDTEVFVTGGGSRLEEELRRRLTEGGGSGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 15 - protein, 779 aa, synthetic construct
  REGION 1..779: note = TFRC
MGSRLEEELRRRLTEGGGSGMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 16 - protein, 364 aa, synthetic construct
  REGION 1..364: note = Alfa-Cetuximab Knob (EGFR)
GEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 17 - protein, 449 aa, synthetic construct
  REGION 1..449: note = Alfa-Cetuximab Hole HC (EGFR)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 214 aa, synthetic construct
  REGION 1..214: note = Alfa-Cetuximab Hole LC (EGFR)
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 489 aa, synthetic construct
  REGION 1..489: note = HA-Cetuximab Knob (EGFR)
AEVKLVESGGGLVKPGGSLKLSCAASGFTFSSYGMSWVRQTPEKRLEWVATISRGGSYTYYPDSVKGRFTISRDNAKNTLYLQMSSLRSEDTAIYYCARRETYDEKGFAYWGQGTTLTVSSGGGGSGGGGSGGGGSDIVLTQSPASLTVSLGQRATISCKSSQSLLNSGNQKNYLTWYQQKPGQPPKLLIYWASTRESGIPARFSGSGSGTDFTLNIHPVEEEDAATYYCQNDNSHPLTFGAGTKLEIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 20 - protein, 449 aa, synthetic construct; identical to SEQ ID NO: 17
  REGION 1..449: note = HA-Cetuximab Hole HC (EGFR)

SEQ ID NO: 21 - protein, 214 aa, synthetic construct; identical to SEQ ID NO: 18
  REGION 1..214: note = HA-Cetuximab Hole LC (EGFR)

SEQ ID NO: 22 - protein, 449 aa, synthetic construct; identical to SEQ ID NO: 17
  REGION 1..449: note = Cetuximab Hole

SEQ ID NO: 23 - protein, 214 aa, synthetic construct; identical to SEQ ID NO: 18
  REGION 1..214: note = Cetuximab Hole

SEQ ID NO: 24 - protein, 455 aa, synthetic construct
  REGION 1..455: note = Zalutumumab Hole
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 214 aa, synthetic construct
  REGION 1..214: note = Zalutumumab Hole
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 449 aa, synthetic construct
  REGION 1..449: note = Panitumumab Hole
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 214 aa, synthetic construct
  REGION 1..214: note = Panitumumab Hole
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 451 aa, synthetic construct
  REGION 1..451: note = Necitumumab Hole
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 214 aa, synthetic construct
  REGION 1..214: note = Necitumumab Hole
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 451 aa, synthetic construct
  REGION 1..451: note = Matuzumab Hole
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 213 aa, synthetic construct
  REGION 1..213: note = Matuzumab Hole
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 459 aa, synthetic construct
  REGION 1..459: note = Trastuzumab Knob HC (anti-HER2)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 33 - protein, 214 aa, synthetic construct
  REGION 1..214: note = Trastuzumab Knob LC (anti-HER2)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 456 aa, synthetic construct
  REGION 1..456: note = Polatuzumab Knob HC (anti-CD79B)
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 35 - protein, 218 aa, synthetic construct
  REGION 1..218: note = Polatuzumab Knob LC (anti-CD79B)
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 460 aa, synthetic construct
  REGION 1..460: note = Belantamab Knob HC (anti-BCMA)
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 37 - protein, 214 aa, synthetic construct
  REGION 1..214: note = Belantamab Knob LC (anti-BCMA)
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 465 aa, synthetic construct
  REGION 1..465: note = Zalutumumab Knob HC (anti-EGFR)
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHHU

SEQ ID NO: 39 - protein, 214 aa, synthetic construct; identical to SEQ ID NO: 25
  REGION 1..214: note = Zalutumumab Knob LC (anti-EGFR)

SEQ ID NO: 40 - protein, 455 aa, synthetic construct
  REGION 1..455: note = Gemtuzumab Knob HC (anti-CD33)
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 41 - protein, 218 aa, synthetic construct
  REGION 1..218: note = Gemtuzumab Knob LC (anti-CD33)
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 460 aa, synthetic construct
  REGION 1..460: note = Inotuzumab Knob HC (anti-CD22)
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 43 - protein, 219 aa, synthetic construct
  REGION 1..219: note = Inotuzumab Knob LC (anti-CD22)
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 459 aa, synthetic construct
  REGION 1..459: note = Loncastuximab Knob HC (anti-CD19)
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 45 - protein, 211 aa, synthetic construct
  REGION 1..211: note = Loncastuximab Knob LC (anti-CD19)
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 460 aa, synthetic construct
  REGION 1..460: note = Sacituzumab Knob HC (anti-TROP2)
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 47 - protein, 214 aa, synthetic construct
  REGION 1..214: note = Sacituzumab Knob LC (anti-TROP2)
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 457 aa, synthetic construct
  REGION 1..457: note = Omburtamab Knob HC (anti-B7-H3)
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 49 - protein, 214 aa, synthetic construct
  REGION 1..214: note = Omburtamab Knob LC (anti-B7-H3)
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 50 - protein, 457 aa, synthetic construct
  REGION 1..457: note = Tisotumab Knob HC (anti-Tissue factor)
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 51 - protein, 214 aa, synthetic construct
  REGION 1..214: note = Tisotumab Knob LC (anti-Tissue factor)
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 458 aa, synthetic construct
  REGION 1..458: note = Farletuzumab Knob HC (anti-FOLR1)
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 53 - protein, 217 aa, synthetic construct
  REGION 1..217: note = Farletuzumab Knob LC (anti-FOLR1)
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 460 aa, synthetic construct
  REGION 1..460: note = Apamistamab Knob HC (anti-CD45)
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 55 - protein, 218 aa, synthetic construct
  REGION 1..218: note = Apamistamab Knob LC (anti-CD45)
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 56 - protein, 920 aa, synthetic construct
  REGION 1..920: note = Serotransferrin Knob (anti-TFRC)
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 57 - protein, 456 aa, synthetic construct
  REGION 1..456: note = Enfortumab (Nectin-4)-4A06 Knob HC (CDCP1)
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 58 - protein, 214 aa, synthetic construct
  REGION 1..214: note = Enfortumab (Nectin-4)-4A06 Knob LC (CDCP1)
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 449 aa, synthetic construct
  REGION 1..449: note = Enfortumab (Nectin-4)-4A06 Hole HC (CDCP1)
EISEVQLVESGGGLVQPGGSLRLSCAASGFNLSYYYIHWVRQAPGKGLEWVASIYSSSSYTSYADSVKGRFTISADTSKNTAYLQMNSLRAEDTAVYYCARAYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 226 aa, synthetic construct
  REGION 1..226: note = Enfortumab (Nectin-4)-4A06 Hole LC (CDCP1)
DIQMTQSPSSLSASVGDRVTITCRASQSVSSAVAWYQQKPGKAPKLLIYSASSLYSGVPSRFSGSRSGTDFTLTISSLQPEDFATYYCQQSYYYYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSDYKDDDDK

SEQ ID NO: 61-62 - skipped (2 entries)

SEQ ID NO: 63 - protein, 466 aa, synthetic construct
  REGION 1..466: note = Enfortumab-Tecentriq Hole HC (PD-L1)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGAWSHPQFEK

SEQ ID NO: 64 - protein, 214 aa, synthetic construct
  REGION 1..214: note = Enfortumab-Tecentriq Hole LC (PD-L1)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65-66 - skipped (2 entries)

SEQ ID NO: 67 - protein, 463 aa, synthetic construct
  REGION 1..463: note = Enfortumab-Trastuzumab Hole HC (HER2)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGAWSHPQFEK

SEQ ID NO: 68 - protein, 214 aa, synthetic construct; identical to SEQ ID NO: 33
  REGION 1..214: note = Enfortumab-Trastuzumab Hole LC (HER2)

SEQ ID NO: 69-70 - skipped (2 entries)

SEQ ID NO: 71 - protein, 449 aa, synthetic construct; identical to SEQ ID NO: 17
  REGION 1..449: note = Enfortumab-Cetuximab Hole HC (EGFR)

SEQ ID NO: 72 - protein, 214 aa, synthetic construct; identical to SEQ ID NO: 18
  REGION 1..214: note = Enfortumab-Cetuximab Hole LC (EGFR)

SEQ ID NO: 73 - protein, 460 aa, synthetic construct; identical to SEQ ID NO: 46
  REGION 1..460: note = Sacituzumab Knob HC (TROP2)

SEQ ID NO: 74 - protein, 214 aa, synthetic construct; identical to SEQ ID NO: 47
  REGION 1..214: note = Sacituzumab Knob LC (TROP2)

(19) United States
(12) Patent Application Publication
(10) Pub. No.: US 2024/0084030 A1
(43) Pub. Date: Mar. 14, 2024

(54) INTERNALIZING RECEPTOR-DIRECTED BISPECIFIC BINDING AGENT-LIGAND FUSIONS FOR THE DEGRADATION OF TARGET PROTEINS

(71) Applicant: The Regents of the University of California, Oakland, CA (US)

(72) Inventors: Katarina PANCE, San Francisco, CA (US); James A. WELLS, San Francisco, CA (US)

(21) Appl. No.: 18/359,562

(22) Filed: Jul. 26, 2023

Related U.S. Application Data
(60) Provisional application No. 63/392,582, filed on Jul. 27, 2022.

(51) Int. Cl. C07K 16/28; A61P 35/00

(52) U.S. Cl. CPC C07K 16/2896; A61P 35/00; C07K 16/2803; C07K 2317/31

(57) ABSTRACT

The present disclosure relates to targeted degradation platform technology. For example, the present disclosure relates to bispecific binding agents for degrading endogenous proteins, whether membrane-associated or soluble, using the lysosome pathway. The disclosure also provides methods useful for producing such agents, nucleic acids encoding same, host cells genetically modified with the nucleic acids, as well as methods for modulating an activity of a cell and/or for the treatment of various disorders.

### CROSS REFERENCE TO RELATED APPLICATION

This application claims the benefit of priority to U.S. Provisional Patent Application No. 63/392,582 filed Jul. 27, 2022, the entire contents of which are incorporated by reference herein and for all purposes.

### STATEMENT REGARDING FEDERALLY-SPONSORED RESEARCH AND DEVELOPMENT

This invention was made with government support under R35 GM122451, and RO1 CA248323 awarded by the National Institutes of Health. The government has certain rights in the invention.

### INCORPORATION OF THE SEQUENCE LISTING

This application contains a Sequence Listing, which is hereby incorporated herein by reference in its entirety. The contents of the electronic sequence listing 2023 Nov. 22 Sequence_Listing_ST26 048536-7380001US.xml; Size: 114,876 bytes; and Date of Creation: Nov. 22, 2023.

### FIELD

The present disclosure relates to targeted degradation platform technology. For example, the present disclosure relates to bispecific binding agents that target internalizing receptors and result in efficient degradation of target proteins, specifically receptors that are traditionally targeted by antibody-drug conjugate (ADC) therapeutics. The disclosure also provides methods useful for producing such agents, nucleic acids encoding same, host cells genetically modified with the nucleic acids, as well as methods for modulating an activity of a cell and/or for the treatment of various disorders.

### BACKGROUND

Targeted protein degradation has emerged in the last two decades as a promising therapeutic modality that has benefits over traditional small molecule or biologic inhibitors. To date, most degraders are heterobifunctional small molecules that recruit intracellular E3 ubiquitin ligases to a target of interest, which induces ubiquitination of the target protein and its subsequent degradation by the proteasome. These have been successful in degrading >60 target proteins and numerous companies have been founded to expand this technology. However, due to their intracellular mechanism of action, these degraders are largely limited to targeting intracellular proteins for degradation. Other technologies have been developed to expand targeted degradation to the extracellular and cell surface proteome. The technologies include AbTACs and ADC-TACs (which co-opt cell surface E3 ligases) and KineTACs (which co-opt cell surface cytokine receptors), which are fully genetically encoded bispecific antibodies. Others are using the internalization of lysosome shuttling receptors M6PR and ASGPR for this purpose. To date, only a handful of receptors have been used for these purposes.

The disclosure provided herein provides internalizing receptors that induce efficient degradation of target proteins, specifically receptors that are traditionally targeted by antibody-drug conjugate (ADC) therapeutics. These receptors are known to internalize rapidly and recycle back to the cell surface, making them ideal degrading receptors. ADCs have targeted these receptors for a different mechanism of action—i.e. delivery of a cytotoxic small molecule inside the cell to selectively kill the target cell. The bispecific antibodies of the disclosure explore a novel mechanism of action for targeting these internalizing receptors—i.e. co-opting their endogenous internalization to induce lysosomal degradation of a target protein.

### BRIEF SUMMARY

The present disclosure demonstrates the development of a new targeted degradation platform technology, which is comprised of fully recombinant bispecific binding agents that utilize internalizing receptor-mediated internalization to target various therapeutically relevant cell surface and extracellular proteins for lysosomal degradation.

Provided herein, among others, includes a bispecific binding agent comprising: a first binding domain that specifically binds to at least one endogenous internalizing receptor, and a second binding domain that specifically binds to a target protein, wherein the internalizing receptor is membrane associated, and wherein the binding of the first binding domain to the at least one internalizing receptor results in the internalization of the target protein bound to the bispecific binding agent.

In some embodiments, the first binding domain specifically binds to one internalizing receptor. In some embodiments, the first binding domain specifically binds to no more than two internalizing receptors. In some embodiments, the at least one endogenous internalizing receptor comprises targeting receptors and recycling receptors. In some embodiments, the at least one endogenous internalizing receptor comprises single-pass and multi-pass membrane proteins.

In one embodiment, the at least one internalizing receptor is selected from the group consisting of HER2, CD30, CD79B, Nectin-4, BCMA, EGFR, CD33, CD20, CD22, CD19, TROP2, B7-H3, Tissue factor, FOLR1, CD45, and TFRC.

In certain embodiments, the binding of the first binding domain to the at least one internalizing receptor results in the degradation of the target protein bound to the bispecific binding agent.

In some embodiments, the target protein comprises a soluble target protein and a membrane-associated target protein. In some embodiments, the target protein is a membrane-associated target protein, and wherein the second binding domain binds to an extracellular epitope of a membrane-associated target protein. In some embodiments, the target cell comprises a neoplastic cell. In some exemplary embodiments, the target cell is a cancer cell selected from the group consisting of breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer. In other embodiments, the target cell comprises an immune cell.

In some embodiments, the target protein is an immune checkpoint protein. In some embodiments, the target protein comprises a cancer antigen. In certain embodiments, the cancer antigen comprises EGFR, CDCP1, CD38, IGF-1R, and MMP14.

In some embodiments, the target protein comprises an immunomodulatory protein. In certain embodiments, the immunomodulatory protein comprises PD-L1, PD-1, CTLA-4, B7-H3, B7-H4, LAG3, NKG2D, TIM-3, VISTA, CD39, CD73 (NT5E), A2AR, SIGLEC7, and SIGLEC15.

In some embodiments, the target protein comprises a soluble target protein. In some embodiments, the soluble target protein comprises an inflammatory cytokine, a growth factor (GF), a toxic enzyme, a target associated with metabolic diseases, a neuronal aggregate, or an autoantibody. In certain embodiments, the inflammatory cytokine comprises lymphotoxin, interleukin-1 (IL-1), IL-2, IL-5, IL-6, IL-12, IL-13, IL-17, IL-18, IL-23, tumor necrosis factor alpha (TNF-α), interferon gamma (IFNγ), and granulocyte-macrophage colony stimulating factor (GM-CSF). In certain embodiments, the growth factor comprises EGF, FGF, NGF, PDGF, VEGF, IGF, GMCSF, GCSF, TGF, RANK-L, erythropieitn, TPO, BMP, HGF, GDF, neurotrophins, MSF, SGF, GDF, and an isoform thereof. In certain embodiments, the toxic enzyme comprises a protein arginine deiminase 1 (PAD1), PAD2, PAD3, PAD4, and PAD6, leucocidin, hemolysin, coagulase, treptokinase, hyaluronidase. In certain embodiments, the toxic enzyme comprises PAD2 or PAD4. In some embodiments, the neuronal aggregate comprises Aβ, TTR, α-synuclein, TAO, and prion. In certain embodiments, the autoantibody comprises IgA, IgE, IgG, IgMand IgD.

In some embodiments, the first binding domain and the second binding domain are each independently selected from the group consisting of natural ligands or a fragment, derivative, or small molecule mimetic thereof, IgG, half antibodies, single-domain antibodies, nanobodies, Fabs, monospecific Fab2, Fc, scFv, minibodies, IgNAR, V-NAR, hcIgG, VHH domain, camelid antibodies, and peptibodies.

In some embodiments, the first binding domain and the second binding domain together form a bispecific antibody, a bispecific diabody, a bispecific Fab2, a bispecific camelid antibody, or a bispecific peptibody, scFv-Fc, a bispecific IgG, a knob and hole bispecific IgG, a Fc-Fab, and a knob and hole bispecific Fc-Fab. In some embodiments, the first binding domain comprises an Fc-Fab, and the second binding domain comprises an Fc-Fab.

In some embodiments, the bispecific binding agent provided herein comprises one or more sequences selected from SEQ ID Nos: 57-74.

Also provided herein incudes a nucleic acid that encodes the bispecific binding agent of the present disclosure. In some embodiments, the nucleic acid is operably connected to a promoter.

Further provided herein incudes an engineered cell capable of protein expression comprising the nucleic acid of the present disclosure. In some embodiments, the engineered cell comprises a B cell, a B memory cell, or a plasma cell.

Another aspect of the present disclosure relates to a method for making a bispecific binding agent provided herein. In some embodiments, the method comprises: i) providing a cell capable of protein synthesis, comprising the nucleic acid disclosed herein and ii) inducing expression of the bispecific binding agent.

The present disclosure further provides a vector which comprises the nucleic acid described herein. In some embodiments, the vector further comprises a promoter, wherein the promoter is operably linked to the nucleic acid.

The present disclosure also provides a pharmaceutical composition. In some embodiments, the pharmaceutical composition comprises the bispecific binding agent, the nucleic acid, the vector, the engineered cell, and a pharmaceutically acceptable excipient.

In another aspect, the present disclosure provides a method of treating a disorder in a subject. In some embodiments, the method comprising administering to a subject in need thereof, a therapeutically effective amount of the bispecific binding agent, the nucleic acid, the vector, the engineered cell, or the pharmaceutical composition provided herein.

In some embodiments, the disorder comprises a neoplastic disorder, an inflammatory disease, a metabolic disorder, an endocrine disorder, and a neurological disorder. In certain embodiments, the neoplastic disorder comprises breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer. In certain embodiments, the inflammatory disease comprises inflammatory intestinal disease, rheumatoid arthritis, lupus, Crohn's disease, and ulcerative colitis. In certain embodiments, the metabolic disorder comprises diabetes, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick, phenylketonuria (PKU), Porphyria, Tay-Sachs disease, and Wilson's disease. In certain embodiments, the neurological disorder comprises Parkinson's disease, Alzheimer's disease, and multiple sclerosis.

In another aspect, the present disclosure comprises a method of degrading a target protein on a surface of a target cell. In some embodiments, the method comprises contacting an endogenous internalizing receptor and the target protein on the surface of the target cell with a binding agent, wherein the binding agent comprises (i) a first binding domain that specifically binds to an endogenous internalizing receptor, wherein the endogenous internalizing receptor comprises Nectin-4 and (ii) a second binding domain that specifically binds to the target protein, wherein the target protein comprises CDCP1. In some embodiments, following the contacting, the target protein is internalized with the endogenous internalizing receptor into the target cell and the target protein is degraded.

In some cases, the binding agent is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the first binding domain binds to an epitope of the endogenous internalizing receptor on the target cell, wherein the epitope comprises at least 80% sequence identity to an epitope to which Enfortumab binds. In some cases, the first binding domain binds to an epitope of the endogenous internalizing receptor on the target cell, wherein the epitope comprises at least 90% sequence identity to the epitope to which Enfortumab binds. In some embodiments, the first binding domain binds to an epitope of the endogenous internalizing receptor on the target cell that does not include any of the amino acids from the epitope to which Enfortumab binds.

In some embodiments, the first binding domain comprises a first binding domain variable heavy chain and a first binding domain variable light chain. In certain embodiments, the first binding domain variable heavy chain comprises at least 80%, sequence identity to SEQ ID NO: 57. In some embodiments, the first binding domain variable heavy chain comprises at least 90%, sequence identity to SEQ ID NO: 57. In some embodiments, the first binding domain variable heavy chain comprises SEQ ID NO: 57. In certain embodiments, the first binding domain variable light chain comprises at least 80% sequence identity to SEQ ID NO: 58. In some embodiments, the first binding domain variable light chain comprises at least 90% sequence identity to SEQ ID NO: 58. In certain embodiments, the first binding domain variable light chain comprises SEQ ID NO: 58.

In some embodiments, the second binding domain comprises a second binding domain variable heavy chain and a second binding domain variable light chain. In some embodiments, the second binding domain variable heavy chain comprises at least 80% sequence identity to SEQ ID NO: 59. In various embodiments, the second binding domain variable heavy chain comprises at least 90% sequence identity to SEQ ID NO: 59. In certain embodiments, the second binding domain variable heavy chain comprises SEQ ID NO: 59. In various embodiments, the second binding domain variable light chain comprises at least 80% sequence identity to SEQ ID NO: 60. In certain embodiments, the second binding domain variable light chain comprises at least 90% sequence identity to SEQ ID NO: 60. In various embodiments, the second binding domain variable light chain comprises SEQ ID NO: 60.

In some cases, the endogenous internalizing receptor is recycled to the target cell surface following the internalization of the binding agent. In various embodiments, the endogenous internalizing receptor is degraded.

In some embodiments, the target cell is a cancer cell. In certain embodiments, the cancer cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, and a colorectal cancer cell.

In some embodiments, expression of CDCP1 on the cancer cell decreases following contact with the bispecific binding agent, as compared to a control cancer cell that is not contacted with the binding agent. In certain embodiments, expression of CDCP1 on the cancer cell decreases by at least 50% or more relative to expression of CDCP1 on the control cancer cell not contacted with the binding agent. In certain embodiments, expression of CDCP1 on the cancer cell decreases by at least 60% or more relative to expression of CDCP1 on the control cancer cell not contacted with the binding agent. In certain embodiments, expression of CDCP1 on the cancer cell decreases by at least 70% or more relative to expression of CDCP1 on the control cancer cell not contacted with the binding agent. In certain embodiments, expression of CDCP1 on the cancer cell decreases by about 50% to 70% or more relative to expression of CDCP1 on the control cancer cell not contacted with the binding agent.

In some embodiments, the method increases the susceptibility of the cancer cell to cancer therapeutic agents. In certain embodiments, the cancer therapeutic agent is a cytotoxic agent. In some embodiments, the method reduces proliferation of the cancer cell. In some cases, the method increases death of the cancer cell. In some embodiments, the contacting is performed in vivo.

In another aspect, the present disclosure provides a method for treating cancer in a subject, the method comprising administering to a subject a binding agent, wherein the binding agent comprises (i) a first binding domain that specifically binds to an endogenous internalizing receptor, wherein the endogenous internalizing receptor is expressed on a target cell, wherein the endogenous internalizing receptor comprises Nectin-4 and (ii) a second binding domain that specifically binds to the target protein, wherein the target protein comprises CDCP1. In some embodiments, the cancer is breast cancer, B cell lymphoma, pancreatic cancer, Hodgkin's lymphoma, ovarian cancer, prostate cancer, mesothelioma, lung cancer, non-Hodgkin's B-cell (B-NHL) lymphoma, melanoma, chronic lymphocytic leukemia, acute lymphocytic leukemia, neuroblastoma, glioma, glioblastoma, bladder cancer, and colorectal cancer. In certain embodiments, the cancer is bladder cancer.

In another aspect, the present disclosure provides a bispecific binding agent comprising (a) a first binding domain that specifically binds to Nectin-4, wherein Nectin-4 is associated with a membrane of a target cell and (b) a second binding domain that specifically binds to a target protein, wherein the target protein is selected from the group consisting of CDCP1, PD-L1, HER2, and EGFR. In some embodiments, the bispecific binding agent is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the first binding domain binds to an epitope of the endogenous internalizing receptor on the target cell, wherein the epitope comprises at least 80% sequence identity to an epitope to which Enfortumab binds. In certain embodiments, the first binding domain binds to an epitope of the endogenous internalizing receptor on the target cell, wherein the epitope comprises at least 90% sequence identity to the epitope to which Enfortumab binds.

In some cases, the first binding domain binds to an epitope of the endogenous internalizing receptor on the target cell that does not include any of the amino acids from the epitope to which Enfortumab binds. In some embodiments, the first binding domain comprises a first binding domain variable heavy chain and a first binding domain variable light chain. In certain embodiments, the first binding domain variable heavy chain comprises at least 80% sequence identity to SEQ ID NO: 57. In some embodiments, the first binding domain variable heavy chain comprises at least 90% sequence identity to SEQ ID NO: 57. In some embodiments, the first binding domain variable heavy chain comprises SEQ ID NO: 57. In some embodiments, the first binding domain variable light chain comprises at least 80% sequence identity to SEQ ID NO: 58. In certain embodiments, the first binding domain variable light chain comprises at least 90% sequence identity to SEQ ID NO: 58. In some embodiments, the first binding domain variable light chain comprises SEQ ID NO: 58.

In some cases, the second binding domain comprises a second binding domain variable heavy chain and a second binding domain variable light chain. In certain embodiments, the second binding domain variable heavy chain comprises at least 80% sequence identity to SEQ ID NO: 59, 63, 67, or 71. In some embodiments, the second binding domain variable heavy chain comprises at least 90% sequence identity to SEQ ID NO: 59, 63, 67, or 71. In certain embodiments, the second binding domain variable heavy chain comprises any of SEQ ID NOs: 59, 63, 67, or 71. In certain embodiments, the second binding domain variable light chain comprises at least 80% sequence identity to any one of SEQ ID NOs: 60, 64, 68, or 72. In some embodiments, the second binding domain variable light chain comprises at least 90% sequence identity to any one of SEQ ID NOs: 60, 64, 68, or 72. In certain embodiments, the second binding domain variable light chain comprises any one of SEQ ID NOs: 60, 64, 68, or 72.

### BRIEF DESCRIPTION OF THE DRAWINGS

FIG. 1 is a schematic of using bispecific antibodies to recruit an internalizing receptor to a target protein of interest, therefore inducing the internalization and lysosomal degradation of the target protein.

FIG. 2 shows a western blot in which dosing of HT-1376 cells for 24 hrs with Enfortumab-4A06 bispecific antibody demonstrates that Nectin-4 can act as a degrading receptor to mediate the degradation of cell surface CDCP1.

### DETAILED DESCRIPTION

The present disclosure provides, among others, fully recombinant bispecific binding agents comprising a first binding domain that specifically binds to at least one endogenous internalizing receptor and a second binding domain for targeted degradation of a target protein, whether soluble or membrane-associated. As used herein, the targeted degradation can be mediated by an internalizing receptor-mediated pathway. In some embodiments, the binding of the first binding domain to the at least one endogenous internalizing receptor results in the internalization of the endogenous internalizing receptor and the bispecific binding agent. In certain embodiments, the endogenous internalizing receptor is membrane associated. Further, the second binding domain can specifically bind to a target protein. The bispecific binding agents of the present disclosure are useful as a targeted degradation platform. The first and second binding domains can be altered and combined for specific purposes.

Targeted protein degradation has emerged over the past two decades as a potential rival to traditional therapeutic modalities for a variety of human diseases. Traditional inhibitors, such as small molecules and biologics, operate through occupancy-driven pharmacology. This paradigm requires high binding potency and frequent dosing to maintain a prolonged therapeutic effect. Furthermore, non-enzymatic protein functions, such as scaffolding functions of kinases, are difficult to block using inhibitors due to lack of ligandable binding areas. Degrader technologies, on the other hand, operate via event-driven pharmacology, enabling one degrader molecule to catalytically degrade multiple target protein molecules. Small molecule degraders, such as PROteolysis TArgeting Chimeras (PROTACs), are heterobifunctional molecules comprised of a ligand to an E3 ubiquitin ligase chemically linked to a protein of interest ligand. Simultaneous binding to both the E3 ligase and target protein enables the transfer of ubiquitin onto the target protein and its subsequent degradation by the proteasome. Small molecule degraders have demonstrated success in degrading over 60 protein targets, providing greater therapeutic benefit compared to the parent inhibitor, overcoming classical resistance mechanisms, and targeting “undruggable” proteins. Furthermore, two PROTACs are currently being tried in phase I clinical trials to test their efficacy and safety as therapeutic agents.

Due to their intracellular mechanism of action, small molecule PROTACs are limited to targeting proteins with cytosolic domains with ligandable surfaces. As such, very few examples exist for PROTACs degrading membrane proteins. Given the vast number of cell surface and extracellular disease-related proteins, there is a critical need to develop degraders capable of targeting this portion of the proteome. Two recent platforms have expanded targeted protein degradation to this important class. One in particular, termed antibody-based PROTACs (AbTACs), utilizes bispecific IgGs to hijack cell surface E3 ligase RNF43 to degrade checkpoint inhibitor protein programmed death-ligand 1 (PD-L1) via the lysosome. The second, termed lysosome-targeting chimeras (LYTACs), utilizes IgG-glycan bioconjugates to co-opt lysosome shuttling receptors, such as mannose-6-phosphate receptor (M6PR) and asialoglycoprotein receptor (ASGPR), to degrade both cell surface and soluble extracellular targets. However, LYTAC production requires complex chemical synthesis and in vitro bioconjugation, thereby limiting the modularity of this platform.

### Definition

The singular form “a”, “an”, and “the” include plural references unless the context clearly dictates otherwise. For example, the term “a cell” includes one or more cells, including mixtures thereof “A and/or B” is used herein to include all of the following alternatives: “A”, “B”, “A or B”, and “A and B.”

The terms “administration” and “administering”, as used interchangeably herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration.

The terms “host cell” and “recombinant cell” are used interchangeably herein. It is understood that such terms, as well as “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the original cell or cell line.

The term “operably linked”, as used herein, denotes a physical or functional linkage between two or more elements, e.g., polypeptide sequences or polynucleotide sequences, which permits them to operate in their intended fashion.

The term “heterologous”, refers to nucleic acid sequences or amino acid sequences operably linked or otherwise joined to one another in a nucleic acid construct or chimeric polypeptide that are not operably linked or are not contiguous to each other in nature.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. This definition also refers to, or may be applied to, the complement of a test sequence. This definition also includes sequences that have deletions and/or additions, as well as those that have substitutions. Sequence identity typically is calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof

The term “treatment” used in reference to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition being treated. Treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention (i.e., reducing the risk of development of clinical symptoms, including causing the clinical symptoms not to develop, e.g., preventing disease progression), and (ii) inhibition (i.e., arresting the development or further development of clinical symptoms, e.g., mitigating or completely inhibiting an active disease).

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” can be a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Although features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

### Compositions of the Disclosure

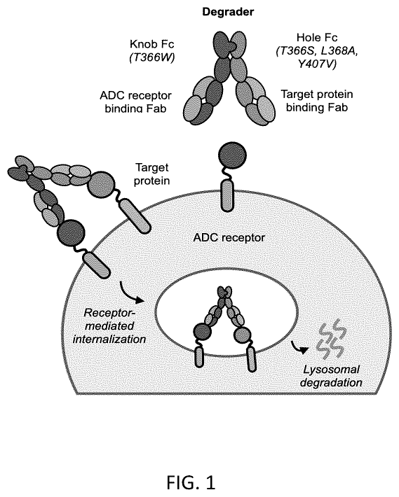

The present disclosure provides, among others, fully recombinant bispecific binding agents comprising a first binding domain that can specifically bind to at least one endogenous internalizing receptor. In some embodiments, the binding of the first binding domain to the at least one endogenous internalizing receptor results in the internalization of the endogenous cell surface receptor and the bispecific binding agent. In other embodiments, the endogenous internalizing receptor can be internalized on its own, and thus results in the internalization of the target protein, which is described in greater detail below, due to simultaneous binding of bispecific binding agent to the endogenous internalizing receptor and target protein. In certain embodiments, the endogenous internalizing receptor is membrane associated. Further, the second binding domain can specifically bind to a target protein. In some non-limiting exemplary embodiments, the present disclosure demonstrates the development of a new targeted degradation platform technology, which includes fully recombinant bispecific binding agents that utilize endogenous internalizing receptor-mediated internalization to target various therapeutically relevant proteins for lysosomal degradation (FIG. 1).

The disclosure also provides, among others, nucleic acids that encode the bispecific binding agents, cells comprising the nucleic acid, and pharmaceutical compositions comprising the bispecific binding agents. The disclosure also provides methods of treatment using bispecific binding agents, nucleic acids encoding bispecific binding agents or pharmaceutical compositions comprising the bispecific binding agents and/or nucleic acids encoding the bispecific binding agents. The disclosure also provides compositions and methods useful for producing such agents, nucleic acids encoding same, host cells genetically modified with the nucleic acids, as well as methods for modulating an activity of a cell and/or for the treatment of various diseases such as cancers.

In some embodiments, the bispecific binding agents are used to treat cancer. In some embodiments, the bispecific binding agents are used to treat solid cancers. In some embodiments, a solid cancer comprises a solid tumor. In some embodiments, the solid cancer comprises bladder cancer.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Bispecific Binding Agents

The bispecific binding agents provided herein comprise a first binding domain and a second binding domain. The first binding domain can specifically bind to at least one endogenous internalizing receptor. In some embodiments, the binding of the first binding domain to the at least one endogenous internalizing receptor results in the internalization of the endogenous internalizing receptor and the bispecific binding agent. In other embodiments, the endogenous internalizing receptor is or can be internalized on its own, and pull in the target protein, which is described in greater detail below, due to simultaneous binding of bispecific binding agent to the endogenous internalizing receptor and target protein. In certain embodiments, the endogenous internalizing receptor is membrane associated. Further, the second binding domain can specifically bind to a target protein.

In some embodiments, the first binding domain of the bispecific binding agents can be a binding agent (e.g., an antibody or a fragment thereof, a peptide, or a small molecule) that binds to the endogenous antibody drug conjugate receptor.

Some compounds that contain binding elements attached to elements that can kill or render cells apoptotic are called antibody-drug conjugates (ADCs). Antibodies are chosen for their ability to selectively target cells with certain ADC receptors common to tumors. See DiJoseph F, Goad M E, Dougher M M, et al. Potent and specific antitumour efficacy of CMC-544, a CD22-targeted immunoconjugate of calicheamicin, against systemically disseminated B cell lymphoma. Clin Cancer Res. 2004; 10:8620-8629. Upon binding of the ADC to the ADC receptor on cells, the ADC-receptor complex is internalized into the cell, where the cytotoxic drug is released. The present disclosure demonstrates that ADC receptor-mediated internalization could be co-opted for targeted degradation applications. ADC receptor is used as its common meaning in the field and refers to a broad category of receptors that can be used as targets for antibody-drug conjugates. Some non-limiting examples of ADC receptors include HER2, CD30, CD79B, Nectin-4, BCMA, EGFR, CD33, CD20, CD22, CD19, TROP2, B7-H3, Tissue factor, FOLR1, CD45, and TFRC.

Exemplary sequences of N-terminally epitope tagged (e.g., alfa, HA, myc) ADC receptors to be targeted include those describe in Table 1 below. Use of epitope tagged receptors allows for identification and validation of the bispecific binding agents of the disclosure using anti-epitope tag primary antibody.

TABLE 1

| Protein | | SEQ |
|---|---|---|
| Name | Sequence | ID NO |
| HER2 | MELAALCRWGLLLALLPPGAASGGSRLEEELRRRLTEG | 1 |
| | GGSGTQVCTGTDMKLRLPASPETHLDMLRHLYQGCQV | |
| | VQGNLELTYLPTNASLSFLQDIQEVQGYVLIAHNQVRQ | |
| | VPLQRLRIVRGTQLFEDNYALAVLDNGDPLNNTTPVTG | |
| | ASPGGLRELQLRSLTEILKGGVLIQRNPQLCYQDTILWK | |
| | DIFHKNNQLALTLIDTNRSRACHPCSPMCKGSRCWGES | |
| | SEDCQSLTRTVCAGGCARCKGPLPTDCCHEQCAAGCTG | |
| | PKHSDCLACLHFNHSGICELHCPALVTYNTDTFESMPNP | |
| | EGRYTFGASCVTACPYNYLSTDVGSCTLVCPLHNQEVT | |
| | AEDGTQRCEKCSKPCARVCYGLGMEHLREVRAVTSAN | |
| | IQEFAGCKKIFGSLAFLPESFDGDPASNTAPLQPEQLQVF | |
| | ETLEEITGYLYISAWPDSLPDLSVFQNLQVIRGRILHNGA | |
| | YSLTLQGLGISWLGLRSLRELGSGLALIHHNTHLCFVHT | |
| | VPWDQLFRNPHQALLHTANRPEDECVGEGLACHQLCA | |
| | RGHCWGPGPTQCVNCSQFLRGQECVEECRVLQGLPRE | |
| | YVNARHCLPCHPECQPQNGSVTCFGPEADQCVACAHY | |
| | KDPPFCVARCPSGVKPDLSYMPIWKFPDEEGACQPCPIN | |
| | CTHSCVDLDDKGCPAEQRASPLTSIISAVVGILLVVVLG | |
| | VVFGILIKRRQQKIRKYTMRRLLQETELVEPLTPSGAMP | |
| | NQAQMRILKETELRKVKVLGSGAFGTVYKGIWIPDGEN | |
| | VKIPVAIKVLRENTSPKANKEILDEAYVMAGVGSPYVS | |
| | RLLGICLTSTVQLVTQLMPYGCLLDHVRENRGRLGSQD | |
| | LLNWCMQIAKGMSYLEDVRLVHRDLAARNVLVKSPN | |
| | HVKITDFGLARLLDIDETEYHADGGKVPIKWMALESILR | |
| | RRFTHQSDVWSYGVTVWELMTFGAKPYDGIPAREIPDL | |
| | LEKGERLPQPPICTIDVYMIMVKCWMIDSECRPRFRELV | |
| | SEFSRMARDPQRFVVIQNEDLGPASPLDSTFYRSLLEDD | |
| | DMGDLVDAEEYLVPQQGFFCPDPAPGAGGMVHHRHRS | |
| | SSTRSGGGDLTLGLEPSEEEAPRSPLAPSEGAGSDVFDG | |
| | DLGMGAAKGLQSLPTHDPSPLQRYSEDPTVPLPSETDG | |
| | YVAPLTCSPQPEYVNQPDVRPQPPSPREGPLPAARPAGA | |
| | TLERPKTLSPGKNGVVKDVFAFGGAVENPEYLTPQGGA | |
| | APQPHPPPAFSPAFDNLYYWDQDPPERGAPPSTFKGTPT | |
| | AENPEYLGLDVPV | |
| CD30 | MRVLLAALGLLFLGALRAGGSRLEEELRRRLTEGGGSG | 2 |
| | FPQDRPFEDTCHGNPSHYYDKAVRRCCYRCPMGLFPTQ | |
| | QCPQRPTDCRKQCEPDYYLDEADRCTACVTCSRDDLVE | |
| | KTPCAWNSSRVCECRPGMFCSTSAVNSCARCFFHSVCP | |
| | AGMIVKFPGTAQKNTVCEPASPGVSPACASPENCKEPSS | |
| | GTIPQAKPTPVSPATSSASTMPVRGGTRLAQEAASKLTR | |
| | APDSPSSVGRPSSDPGLSPTQPCPEGSGDCRKQCEPDYY | |
| | LDEAGRCTACVSCSRDDLVEKTPCAWNSSRTCECRPG | |
| | MICATSATNSCARCVPYPICAAETVTKPQDMAEKDTTF | |
| | EAPPLGTQPDCNPTPENGEAPASTSPTQSLLVDSQASKT | |
| | LPIPTSAPVALSSTGKPVLDAGPVLFWVILVLVVVVGSS | |
| | AFLLCHRRACRKRIRQKLHLCYPVQTSQPKLELVDSRPR | |
| | RSSTQLRSGASVTEPVAEERGLMSQPLMETCHSVGAAY | |
| | LESLPLQDASPAGGPSSPRDLPEPRVSTEHTNNKIEKIYI | |
| | MKADTVIVGTVKAELPEGRGLAGPAEPELEEELEADHT | |
| | PHYPEQETEPPLGSCSDVMLSVEEEGKEDPLPTAASGK | |
| CD79B | MARLALSPVPSHWMVALLLLLSAEPVPAGGSRLEEELR | 3 |
| | RRLTEGGGSGARSEDRYRNPKGSACSRIWQSPRFIARKR | |
| | GFTVKMHCYMNSASGNVSWLWKQEMDENPQQLKLEK | |
| | GRMEESQNESLATLTIQGIRFEDNGIYFCQQKCNNTSEV | |
| | YQGCGTELRVMGFSTLAQLKQRNTLKDGIIMIQTLLIILF | |
| | IIVPIFLLLDKDDSKAGMEEDHTYEGLDIDQTATYEDIVT | |
| | LRTGEVKWSVGEHPGQE | |
| Nectin-4 | MPLSLGAEMWGPEAWLLLLLLLASFTGRCPAGGSRLEE | 4 |
| | ELRRRLTEGGGSGGELETSDVVTVVLGQDAKLPCFYRG | |
| | DSGEQVGQVAWARVDAGEGAQELALLHSKYGLHVSP | |
| | AYEGRVEQPPPPRNPLDGSVLLRNAVQADEGEYECRVS | |
| | TFPAGSFQARLRLRVLVPPLPSLNPGPALEEGQGLTLAA | |
| | SCTAEGSPAPSVTWDTEVKGTTSSRSFKHSRSAAVTSEF | |
| | HLVPSRSMNGQPLTCVVSHPGLLQDQRITHILHVSFLAE | |
| | ASVRGLEDQNLWHIGREGAMLKCLSEGQPPPSYNWTR | |
| | LDGPLPSGVRVDGDTLGFPPLTTEHSGIYVCHVSNEFSS | |
| | RDSQVTVDVLDPQEDSGKQVDLVSASVVVVGVIAALLF | |
| | CLLVVVVVLMSRYHRRKAQQMTQKYEEELTLTRENSI | |
| | RRLHSHHTDPRSQPEESVGLRAEGHPDSLKDNSSCSVM | |
| | SEEPEGRSYSTLTTVREIETQTELLSPGSGRAEEEEDQDE | |
| | GIKQAMNHFVQENGTLRAKPTGNGIYINGRGHLV | |
| BCMA | MGSRLEEELRRRLTEGGGSGLQMAGQCSQNEYFDSLLH | 5 |
| | ACIPCQLRCSSNTPPLTCQRYCNASVTNSVKGTNAILWT | |
| | CLGLSLIISLAVFVLMFLLRKINSEPLKDEFKNTGSGLLG | |
| | MANIDLEKSRTGDEIILPRGLEYTVEECTCEDCIKSKPKV | |
| | DSDHCFPLPAMEEGATILVTTKTNDYCKSLPAALSATEI | |
| | EKSISAR | |
| EGFR | MRPSGTAGAALLALLAALCPASRAGGSRLEEELRRRLT | 6 |
| | EGGGSGLEEKKVCQGTSNKLTQLGTFEDHFLSLQRMFN | |
| | NCEVVLGNLEITYVQRNYDLSFLKTIQEVAGYVLIALNT | |
| | VERIPLENLQIIRGNMYYENSYALAVLSNYDANKTGLK | |
| | ELPMRNLQEILHGAVRFSNNPALCNVESIQWRDIVSSDF | |
| | LSNMSMDFQNHLGSCQKCDPSCPNGSCWGAGEENCQK | |
| | LTKIICAQQCSGRCRGKSPSDCCHNQCAAGCTGPRESDC | |
| | LVCRKFRDEATCKDTCPPLMLYNPTTYQMDVNPEGKY | |
| | SFGATCVKKCPRNYVVTDHGSCVRACGADSYEMEEDG | |
| | VRKCKKCEGPCRKVCNGIGIGEFKDSLSINATNIKHFKN | |
| | CTSISGDLHILPVAFRGDSFTHTPPLDPQELDILKTVKEIT | |
| | GFLLIQAWPENRTDLHAFENLEIIRGRTKQHGQFSLAVV | |
| | SLNITSLGLRSLKEISDGDVIISGNKNLCYANTINWKKLF | |
| | GTSGQKTKIISNRGENSCKATGQVCHALCSPEGCWGPE | |
| | PRDCVSCRNVSRGRECVDKCNLLEGEPREFVENSECIQC | |
| | HPECLPQAMNITCTGRGPDNCIQCAHYIDGPHCVKTCP | |
| | AGVMGENNTLVWKYADAGHVCHLCHPNCTYGCTGPG | |
| | LEGCPTNGPKIPSIATGMVGALLLLLVVALGIGLFMRRR | |
| | HIVRKRTLRRLLQERELVEPLTPSGEAPNQALLRILKETE | |
| | FKKIKVLGSGAFGTVYKGLWIPEGEKVKIPVAIKELREA | |
| | TSPKANKEILDEAYVMASVDNPHVCRLLGICLTSTVQLI | |
| | TQLMPFGCLLDYVREHKDNIGSQYLLNWCVQIAKGMN | |
| | YLEDRRLVHRDLAARNVLVKTPQHVKITDFGLAKLLG | |
| | AEEKEYHAEGGKVPIKWMALESILHRIYTHQSDVWSYG | |
| | VTVWELMTFGSKPYDGIPASEISSILEKGERLPQPPICTID | |
| | VYMIMVKCWMIDADSRPKFRELIIEFSKMARDPQRYLV | |
| | IQGDERMHLPSPTDSNFYRALMDEEDMDDVVDADEYLI | |
| | PQQGFFSSPSTSRTPLLSSLSATSNNSTVACIDRNGLQSC | |
| | PIKEDSFLQRYSSDPTGALTEDSIDDTFLPVPEYINQSVP | |
| | KRPAGSVQNPVYHNQPLNPAPSRDPHYQDPHSTAVGNP | |
| | EYLNTVQPTCVNSTFDSPAHWAQKGSHQISLDNPDYQQ | |
| | DFFPKEAKPNGIFKGSTAENAEYLRVAPQSSEFIGA | |
| CD33 | MPLLLLLPLLWAGALAMGGSRLEEELRRRLTEGGGSGD | 7 |
| | PNFWLQVQESVTVQEGLCVLVPCTFFHPIPYYDKNSPV | |
| | HGYWFREGAIISRDSPVATNKLDQEVQEETQGRFRLLG | |
| | DPSRNNCSLSIVDARRRDNGSYFFRMERGSTKYSYKSP | |
| | QLSVHVTDLTHRPKILIPGTLEPGHSKNLTCSVSWACEQ | |
| | GTPPIFSWLSAAPTSLGPRTTHSSVLIITPRPQDHGTNLTC | |
| | QVKFAGAGVTTERTIQLNVTYVPQNPTTGIFPGDGSGK | |
| | QETRAGVVHGAIGGAGVTALLALCLCLIFFIVKTHRRK | |
| | AARTAVGRNDTHPTTGSASPKHQKKSKLHGPTETSSCS | |
| | GAAPTVEMDEELHYASLNFHGMNPSKDTSTEYSEVRTQ | |
| CD20 | MGSRLEEELRRRLTEGGGSGTTPRNSVNGTFPAEPMKG | 8 |
| | PIAMQSGPKPLFRRMSSLVGPTQSFFMRESKTLGAVQIM | |
| | NGLFHIALGGLLMIPAGIYAPICVTVWYPLWGGIMYIIS | |
| | GSLLAATEKNSRKCLVKGKMIMNSLSLFAAISGMILSIM | |
| | DILNIKISHFLKMESLNFIRAHTPYINIYNCEPANPSEKNS | |
| | PSTQYCYSIQSLFLGILSVMLIFAFFQELVIAGIVENEWK | |
| | RTCSRPKSNIVLLSAEEKKEQTIEIKEEVVGLTETSSQPK | |
| | NEEDIEIIPIQEEEEEETETNFPEPPQDQESSPIENDSSP | |
| CD22 | MHLLGPWLLLLVLEYLAFSGGSRLEEELRRRLTEGGGS | 9 |
| | GDSSKWVFEHPETLYAWEGACVWIPCTYRALDGDLES | |
| | FILFHNPEYNKNTSKFDGTRLYESTKDGKVPSEQKRVQF | |
| | LGDKNKNCTLSIHPVHLNDSGQLGLRMESKTEKWMERI | |
| | HLNVSERPFPPHIQLPPEIQESQEVTLTCLLNFSCYGYPIQ | |
| | LQWLLEGVPMRQAAVTSTSLTIKSVFTRSELKFSPQWS | |
| | HHGKIVTCQLQDADGKFLSNDTVQLNVKHTPKLEIKVT | |
| | PSDAIVREGDSVTMTCEVSSSNPEYTTVSWLKDGTSLK | |
| | KQNTFTLNLREVTKDQSGKYCCQVSNDVGPGRSEEVFL | |
| | QVQYAPEPSTVQILHSPAVEGSQVEFLCMSLANPLPTNY | |
| | TWYHNGKEMQGRTEEKVHIPKILPWHAGTYSCVAENIL | |
| | GTGQRGPGAELDVQYPPKKVTTVIQNPMPIREGDTVT | |
| | LSCNYNSSNPSVTRYEWKPHGAWEEPSLGVLKIQNVG | |
| | WDNTTIACAACNSWCSWASPVALNVQYAPRDVRVRKI | |
| | KPLSEIHSGNSVSLQCDFSSSHPKEVQFFWEKNGRLLGK | |
| | ESQLNFDSISPEDAGSYSCWVNNSIGQTASKAWTLEVL | |
| | YAPRRLRVSMSPGDQVMEGKSATLTCESDANPPVSHYT | |
| | WFDWNNQSLPYHSQKLRLEPVKVQHSGAYWCQGTNS | |
| | VGKGRSPLSTLTVYYSPETIGRRVAVGLGSCLAILILAIC | |
| | GLKLQRRWKRTQSQQGLQENSSGQSFFVRNKKVRR | |
| | APLSEGPHSLGCYNPMMEDGISYTTLRFPEMNIPRTGDA | |
| | ESSEMQRPPPDCDDTVTYSALHKRQVGDYENVIPDFPE | |
| | DEGIHYSELIQFGVGERPQAQENVDYVILKH | |
| CD19 | MPPPRLLFFLLFLTPMEVRGGSRLEEELRRRLTEGGGSG | 10 |
| | PEEPLVVKVEEGDNAVLQCLKGTSDGPTQQLTWSRESP | |
| | LKPFLKLSLGLPGLGIHMRPLAIWLFIFNVSQQMGGFYL | |
| | CQPGPPSEKAWQPGWTVNVEGSGELFRWNVSDLGGLG | |
| | CGLKNRSSEGPSSPSGKLMSPKLYVWAKDRPEIWEGEP | |
| | PCLPPRDSLNQSLSQDLTMAPGSTLWLSCGVPPDSVSRG | |
| | PLSWTHVHPKGPKSLLSLELKDDRPARDMWVMETGLL | |
| | LPRATAQDAGKYYCHRGNLTMSFHLEITARPVLWHWL | |
| | LRTGGWKVSAVTLAYLIFCLCSLVGILHLQRALVLRRK | |
| | RKRMTDPTRRFFKVTPPPGSGPQNQYGNVLSLPTPTSGL | |
| | GRAQRWAAGLGGTAPSYGNPSSDVQADGALGSRSPPG | |
| | VGPEEEEGEGYEEPDSEEDSEFYENDSNLGQDQLSQDG | |
| | SGYENPEDEPLGPEDEDSFSNAESYENEDEELTQPVART | |
| | MDFLSPHGSAWDPSREATSLGSQSYEDMRGILYAAPQL | |
| | RSIRGQPGPNHEEDADSYENMDNPDGPDPAWGGGGRM | |
| | GTWSTR | |
| TROP2 | MARGPGLAPPPLRLPLLLLVLAAVTGGGSRLEEELRRRL | 11 |
| | TEGGGSGHTAAQDNCTCPTNKMTVCSPDGPGGRCQCR | |
| | ALGSGMAVDCSTLTSKCLLLKARMSAPKNARTLVRPSE | |
| | HALVDNDGLYDPDCDPEGRFKARQCNQTSVCWCVNSV | |
| | GVRRTDKGDLSLRCDELVRTHHILIDLRHRPTAGAFNHS | |
| | DLDAELRRLFRERYRLHPKFVAAVHYEQPTIQIELRQNT | |
| | SQKAAGDVDIGDAAYYFERDIKGESLFQGRGGLDLRVR | |
| | GEPLQVERTLIYYLDEIPPKFSMKRLTAGLIAVIVVVVV | |
| | ALVAGMAVLVITNRRKSGKYKKVEIKELGELRKEPSL | |
| B7-H3 | MLRRRGSPGMGVHVGAALGALWFCLTGAGGSRLEEEL | 12 |
| | RRRLTEGGGSGLEVQVPEDPVVALVGTDATLCCSFSPEP | |
| | GFSLAQLNLIWQLTDTKQLVHSFAEGQDQGSAYANRT | |
| | ALFPDLLAQGNASLRLQRVRVADEGSFTCFVSIRDFGSA | |
| | AVSLQVAAPYSKPSMTLEPNKDLRPGDTVTITCSSYQG | |
| | YPEAEVFWQDGQGVPLTGNVTTSQMANEQGLFDVHSI | |
| | LRVVLGANGTYSCLVRNPVLQQDAHSSVTITPQRSPTG | |
| | AVEVQVPEDPVVALVGTDATLRCSFSPEPGFSLAQLNLI | |
| | WQLTDTKQLVHSFTEGRDQGSAYANRTALFPDLLAQG | |
| | NASLRLQRVRVADEGSFTCFVSIRDFGSAAVSLQVAAP | |
| | YSKPSMTLEPNKDLRPGDTVTITCSSYRGYPEAEVFWQ | |
| | DGQGVPLTGNVTTSQMANEQGLFDVHSVLRVVLGANG | |
| | TYSCLVRNPVLQQDAHGSVTITGQPMTFPPEALWVTVG | |
| | LSVCLIALLVALAFVCWRKIKQSCEEENAGAEDQDGEG | |
| | EGSKTALQPLKHSDSKEDDGQEIA | |
| FOLR1 | MAQRMTTQLLLLLVWVAVVGEAQTGGSRLEEELRRRL | 13 |
| | TEGGGSGRIAWARTELLNVCMNAKHHKEKPGPEDKLH | |
| | EQCRPWRKNACCSTNTSQEAHKDVSYLYRFNWNHCGE | |
| | MAPACKRHFIQDTCLYECSPNLGPWIQQVDQSWRKER | |
| | VLNVPLCKEDCEQWWEDCRTSYTCKSNWHKGWNWTS | |
| | GFNKCAVGAACQPFHFYFPTPTVLCNEIWTHSYKVSNY | |
| | SRGSGRCIQMWFDPAQGNPNEEVARFYAAAMS | |
| CD45 | MTMYLWLKLLAFGFAFLDTEVFVTGGGSRLEEELRRRL | 14 |
| | TEGGGSGQSPTPSPTGLTTAKMPSVPLSSDPLPTHTTAFS | |
| | PASTFERENDFSETTTSLSPDNTSTQVSPDSLDNASAFNT | |
| | TGVSSVQTPHLPTHADSQTPSAGTDTQTFSGSAANAKL | |
| | NPTPGSNAISDVPGERSTASTFPTDPVSPLTTTLSLAHHS | |
| | SAALPARTSNTTITANTSDAYLNASETTTLSPSGSAVIST | |
| | TTIATTPSKPTCDEKYANITVDYLYNKETKLFTAKLNVN | |
| | ENVECGNNTCTNNEVHNLTECKNASVSISHNSCTAPDK | |
| | TLILDVPPGVEKFQLHDCTQVEKADTTICLKWKNIETFT | |
| | CDTQNITYRFQCGNMIFDNKEIKLENLEPEHEYKCDSEI | |
| | LYNNHKFTNASKIIKTDFGSPGEPQIIFCRSEAAHQGVIT | |
| | WNPPQRSFHNFTLCYIKETEKDCLNLDKNLIKYDLQNL | |
| | KPYTKYVLSLHAYIIAKVQRNGSAAMCHFTTKSAPPSQ | |
| | VWNMTVSMTSDNSMHVKCRPPRDRNGPHERYHLEVE | |
| | AGNTLVRNESHKNCDFRVKDLQYSTDYTFKAYFHNGD | |
| | YPGEPFILHHSTSYNSKALIAFLAFLIIVTSIALLVVLYKI | |
| | YDLHKKRSCNLDEQQELVERDDEKQLMNVEPIHADILL | |
| | ETYKRKIADEGRLFLAEFQSIPRVFSKFPIKEARKPFNQN | |
| | KNRYVDILPYDYNRVELSEINGDAGSNYINASYIDGFKE | |
| | PRKYIAAQGPRDETVDDFWRMIWEQKATVIVMVTRCE | |
| | EGNRNKCAEYWPSMEEGTRAFGDVVVKINQHKRCPDY | |
| | IIQKLNIVNKKEKATGREVTHIQFTSWPDHGVPEDPHLL | |
| | LKLRRRVNAFSNFFSGPIVVHCSAGVGRTGTYIGIDAML | |
| | EGLEAENKVDVYGYVVKLRRQRCLMVQVEAQYILIHQ | |
| | ALVEYNQFGETEVNLSELHPYLHNMKKRDPPSEPSPLE | |
| | AEFQRLPSYRSWRTQHIGNQEENKSKNRNSNVIPYDYN | |
| | RVPLKHELEMSKESEHDSDESSDDDSDSEEPSKYINASFI | |
| | MSYWKPEVMIAAQGPLKETIGDFWQMIFQRKVKVIVM | |
| | LTELKHGDQEICAQYWGEGKQTYGDIEVDLKDTDKSST | |
| | YTLRVFELRHSKRKDSRTVYQYQYTNWSVEQLPAEPKE | |
| | LISMIQVVKQKLPQKNSSEGNKHHKSTPLLIHCRDGSQQ | |
| | TGIFCALLNLLESAETEEVVDIFQVVKALRKARPGMVST | |
| | FEQYQFLYDVIASTYPAQNGQVKKNNHQEDKIEFDNEV | |
| | DKVKQDANCVNPLGAPEKLPEAKEQAEGSEPTSGTEGP | |
| | EHSVNGPASPALNQGS | |
| TFRC | MGSRLEEELRRRLTEGGGSGMDQARSAFSNLFGGEPLS | 15 |
| | YTRFSLARQVDGDNSHVEMKLAVDEEENADNNTKAN | |
| | VTKPKRCSGSICYGTIAVIVFFLIGFMIGYLGYCKGVEPK | |
| | TECERLAGTESPVREEPGEDFPAARRLYWDDLKRKLSE | |
| | KLDSTDFTGTIKLLNENSYVPREAGSQKDENLALYVEN | |
| | QFREFKLSKVWRDQHFVKIQVKDSAQNSVIIVDKNGRL | |
| | VYLVENPGGYVAYSKAATVTGKLVHANFGTKKDFEDL | |
| | YTPVNGSIVIVRAGKITFAEKVANAESLNAIGVLIYMDQ | |
| | TKFPIVNAELSFFGHAHLGTGDPYTPGFPSFNHTQFPPSR | |
| | SSGLPNIPVQTISRAAAEKLFGNMEGDCPSDWKTD | |
| | STCRMVTSESKNVKLTVSNVLKEIKILNIFGVIKGFVEPD | |
| | HYVVVGAQRDAWGPGAAKSGVGTALLLKLAQMFSDM | |
| | VLKDGFQPSRSIIFASWSAGDFGSVGATEWLEGYLSSLH | |
| | LKAFTYINLDKAVLGTSNFKVSASPLLYTLIEKTMQNVK | |
| | HPVTGQFLYQDSNWASKVEKLTLDNAAFPFLAYSGIPA | |
| | VSFCFCEDTDYPYLGTTMDTYKELIERIPELNKVARAAA | |
| | EVAGQFVIKLTHDVELNLDYERYNSQLLSFVRDLNQYR | |
| | ADIKEMGLSLQWLYSARGDFFRATSRLTTDFGNAEKTD | |
| | RFVMKKLNDRVMRVEYHFLSPYVSPKESPFRHVFWGS | |
| | GSHTLPALLENLKLRKQNNGAFNETLFRNQLALATWTI | |
| | QGAANALSGDVWDIDNEF | |

In a proof-of-concept example, as described infra, enfortumab is used as the first binding domain of the bispecific binding agent provided herein. Enfortumab is known to specifically bind to the ADC receptor, Nectin-4. Binding to Nectin-4 leads to its internalization and shuttling to the lysosome for degradation. In some non-limiting exemplary embodiments, the present disclosure demonstrates the development of a new targeted degradation platform technology, which comprise of fully recombinant bispecific binding agents that utilize internalizing receptor-mediated internalization of its cognate receptors to target various therapeutically relevant cell surface proteins for lysosomal degradation.

The first binding domain can specifically bind to at least one internalizing receptor. The first binding domain of the bispecific binding agents provided herein can specifically bind to one or more cell surface receptors. In some embodiments, the first binding domain specifically binds to one internalizing receptor. In some embodiments, the first binding domain specifically binds to no more than two internalizing receptors. In some embodiments, the first binding domain specifically binds to two internalizing receptors. In some embodiments, the internalizing surface receptor can be a monomeric receptor. In some embodiments, the internalizing receptor can form a complex with other molecules.

The internalizing receptors can be internalizing receptors or internalizing and recycling receptors. An internalizing receptor as used herein refers to an internalizing receptor that specifically binds to a ligand (e.g., a cytokine, growth factor or an isoform or a derivative capable of binding thereof), and such binding results in internalization and degradation. In some embodiments, degradation can occur through delivery of the target protein discussed herein to a lysosome via the internalizing receptor. In contrast, an internalizing and recycling receptor as used herein refers to an internalizing receptor that specifically binds to a ligand, e.g., a cytokine, a chemokine, an antibody or fragment thereof, a growth factor or an isoform or a derivative capable of binding thereof, and leads to internalization but the receptor itself is not degraded.

In some embodiments, the binding of the first binding domain to the at least one internalizing receptor results in the internalization of the internalizing receptor and the bispecific binding agent. In some embodiments, the binding of the first binding domain to the at least one internalizing receptor results in the degradation of the target protein bound to the bispecific binding agent described herein. In certain embodiments, the binding of the first binding domain to at least one internalizing receptor results in the degradation of the target protein bound to the bispecific binding agent described herein, but not the bispecific binding agent.

In certain embodiments, the internalizing receptor is membrane associated. Membrane proteins represent about a third of the proteins in living organisms and many membrane proteins are known in the field. Based on their structure, membrane proteins can be largely categorized into three main types: (1) integral membrane protein (IMP), which is permanently anchored or part of the membrane, (2) peripheral membrane protein, which is temporarily attached to the lipid bilayer or to other integral proteins, and (3) lipid-anchored proteins. The most common type of IMP is the transmembrane protein (TM), which spans the entire biological membrane. The internalizing receptor of the present disclosure include single-pass and multi-pass membrane proteins. Single-pass membrane proteins cross the membrane only once, while multi-pass membrane proteins weave in and out, crossing several times.

In some embodiments, the first binding domain can be an antigen-binding domain from any antigen-binding molecules, such as any of the clinically approved antibodies, known or to be developed. Some exemplary therapeutic monoclonal antibodies approved or in review in the EU or US are provided in Table 2 below.

TABLE 2

| Protein Name | Sequence | SEQ ID NO |
|---|---|---|
| Alfa-Cetuximab | Knob: | 16 |
| (EGFR) | GEVQLQESGGGLVQPGGSLRLSCTASGVTISAL | |
| | NAMAMGWYRQAPGERRVMVAAVSERGNAM | |
| | YRESVQGRFTVTRDFTNKMVSLQMDNLKPEDT | |
| | AVYYCHVLEDRVDSFHDYWGQGTQVTVSSEP | |
| | KSCDKTHTCPPCPAPELLGGPSVFLFPPKPKDTL | |
| | MISRTPEVTCVVVDVSHEDPEVKFNWYVDGVE | |
| | VHNAKTKPREEQYNSTYRVVSVLTVLHQDWL | |
| | NGKEYKCKVSNKALPAPIEKTISKAKGQPREPQ | |
| | VYTLPPSRDELTKNQVSLWCLVKGFYPSDIAVE | |
| | WESNGQPENNYKTTPPVLDSDGSFFLYSKLTV | |
| | DKSRWQQGNVFSCSVMHEALHNHYTQKSLSL | |
| | SPGKGGSHHHHHH | |
| | Hole HC: | 17 |
| | QVQLKQSGPGLVQPSQSLSITCTVSGFSLTNYG | |
| | VHWVRQSPGKGLEWLGVIWSGGNTDYNTPFT | |
| | SRLSINKDNSKSQVFFKMNSLQSNDTAIYYCAR | |
| | ALTYYDYEFAYWGQGTLVTVSAASTKGPSVFP | |
| | LAPSSKSTSGGTAALGCLVKDYFPEPVTVSWNS | |
| | GALTSGVHTFPAVLQSSGLYSLSSVVTVPSSSL | |
| | GTQTYICNVNHKPSNTKVDKKVEPKSCDKTHT | |
| | CPPCPAPELLGGPSVFLFPPKPKDTLMISRTPEV | |
| | TCVVVDVSHEDPEVKFNWYVDGVEVHNAKTK | |
| | PREEQYNSTYRVVSVLTVLHQDWLNGKEYKC | |
| | KVSNKALPAPIEKTISKAKGQPREPQVYTLPPSR | |
| | DELTKNQVSLSCAVKGFYPSDIAVEWESNGQP | |
| | ENNYKTTPPVLDSDGSFFLVSKLTVDKSRWQQ | |
| | GNVFSCSVMHEALHNHYTQKSLSLSPGK | |
| HA-Cetuximab | Hole LC: | 18 |
| (EGFR) | DILLTQSPVILSVSPGERVSFSCRASQSIGTNIHW | |
| | YQQRTNGSPRLLIKYASESISGIPSRFSGSGSGTD | |
| | FTLSINSVESEDIADYYCQQNNNWPTTFGAGTK | |
| | LELKRTVAAPSVFIFPPSDEQLKSGTASVVCLLN | |
| | NFYPREAKVQWKVDNALQSGNSQESVTEQDS | |
| | KDSTYSLSSTLTLSKADYEKHKVYACEVTHQG | |
| | LSSPVTKSFNRGEC | |
| | Knob: | 19 |
| | AEVKLVESGGGLVKPGGSLKLSCAASGFTFSSY | |
| | GMSWVRQTPEKRLEWVATISRGGSYTYYPDSV | |
| | KGRFTISRDNAKNTLYLQMSSLRSEDTAIYYCA | |
| | RRETYDEKGFAYWGQGTTLTVSSGGGGSGGG | |
| | GSGGGGSDIVLTQSPASLTVSLGQRATISCKSSQ | |
| | SLLNSGNQKNYLTWYQQKPGQPPKLLIYWAST | |
| | RESGIPARFSGSGSGTDFTLNIHPVEEEDAATYY | |
| | CQNDNSHPLTFGAGTKLEIEPKSCDKTHTCPPC | |
| | PAPELLGGPSVFLFPPKPKDTLMISRTPEVTCVV | |
| | VDVSHEDPEVKFNWYVDGVEVHNAKTKPREE | |
| | QYNSTYRVVSVLTVLHQDWLNGKEYKCKVSN | |
| | KALPAPIEKTISKAKGQPREPQVYTLPPSRDELT | |
| | KNQVSLWCLVKGFYPSDIAVEWESNGQPENNY | |
| | KTTPPVLDSDGSFFLYSKLTVDKSRWQQGNVF | |
| | SCSVMHEALHNHYTQKSLSLSPGKGGSHHHHH | |
| | H | |
| | Hole HC: | 20 |
| | QVQLKQSGPGLVQPSQSLSITCTVSGFSLTNYG | |
| | VHWVRQSPGKGLEWLGVIWSGGNTDYNTPFT | |
| | SRLSINKDNSKSQVFFKMNSLQSNDTAIYYCAR | |
| | ALTYYDYEFAYWGQGTLVTVSAASTKGPSVFP | |
| | LAPSSKSTSGGTAALGCLVKDYFPEPVTVSWNS | |
| | GALTSGVHTFPAVLQSSGLYSLSSVVTVPSSSL | |
| | GTQTYICNVNHKPSNTKVDKKVEPKSCDKTHT | |
| | CPPCPAPELLGGPSVFLFPPKPKDTLMISRTPEV | |
| | TCVVVDVSHEDPEVKFNWYVDGVEVHNAKTK | |
| | PREEQYNSTYRVVSVLTVLHQDWLNGKEYKC | |
| | KVSNKALPAPIEKTISKAKGQPREPQVYTLPPSR | |
| | DELTKNQVSLSCAVKGFYPSDIAVEWESNGQP | |
| | ENNYKTTPPVLDSDGSFFLVSKLTVDKSRWQQ | |
| | GNVFSCSVMHEALHNHYTQKSLSLSPGK | |
| | Hole LC: | 21 |
| | DILLTQSPVILSVSPGERVSFSCRASQSIGTNIHW | |
| | YQQRTNGSPRLLIKYASESISGIPSRFSGSGSGTD | |
| | FTLSINSVESEDIADYYCQQNNNWPTTFGAGTK | |
| | LELKRTVAAPSVFIFPPSDEQLKSGTASVVCLLN | |
| | NFYPREAKVQWKVDNALQSGNSQESVTEQDS | |
| | KDSTYSLSSTLTLSKADYEKHKVYACEVTHQG | |
| | LSSPVTKSFNRGEC | |
| Cetuximab hole | HC: | 22 |
| | QVQLKQSGPGLVQPSQSLSITCTVSGFSLTNYG | |
| | VHWVRQSPGKGLEWLGVIWSGGNTDYNTPFT | |
| | SRLSINKDNSKSQVFFKMNSLQSNDTAIYYCAR | |
| | ALTYYDYEFAYWGQGTLVTVSAASTKGPSVFP | |
| | LAPSSKSTSGGTAALGCLVKDYFPEPVTVSWNS | |
| | GALTSGVHTFPAVLQSSGLYSLSSVVTVPSSSL | |
| | GTQTYICNVNHKPSNTKVDKKVEPKSCDKTHT | |
| | CPPCPAPELLGGPSVFLFPPKPKDTLMISRTPEV | |
| | TCVVVDVSHEDPEVKFNWYVDGVEVHNAKTK | |
| | PREEQYNSTYRVVSVLTVLHQDWLNGKEYKC | |
| | KVSNKALPAPIEKTISKAKGQPREPQVYTLPPSR | |
| | DELTKNQVSLSCAVKGFYPSDIAVEWESNGQP | |
| | ENNYKTTPPVLDSDGSFFLVSKLTVDKSRWQQ | |
| | GNVFSCSVMHEALHNHYTQKSLSLSPGK | |
| | LC: | 23 |
| | DILLTQSPVILSVSPGERVSFSCRASQSIGTNIHW | |
| | YQQRTNGSPRLLIKYASESISGIPSRFSGSGSGTD | |
| | FTLSINSVESEDIADYYCQQNNNWPTTFGAGTK | |
| | LELKRTVAAPSVFIFPPSDEQLKSGTASVVCLLN | |
| | NFYPREAKVQWKVDNALQSGNSQESVTEQDS | |
| | KDSTYSLSSTLTLSKADYEKHKVYACEVTHQG | |
| | LSSPVTKSFNRGEC | |
| Zalutumumab | HC: | 24 |
| hole | QVQLVESGGGVVQPGRSLRLSCAASGFTFSTY | |
| | GMHWVRQAPGKGLEWVAVIWDDGSYKYYGD | |
| | SVKGRFTISRDNSKNTLYLQMNSLRAEDTAVY | |
| | YCARDGITMVRGVMKDYFDYWGQGTLVTVSS | |
| | ASTKGPSVFPLAPSSKSTSGGTAALGCLVKDYF | |
| | PEPVTVSWNSGALTSGVHTFPAVLQSSGLYSLS | |
| | SVVTVPSSSLGTQTYICNVNHKPSNTKVDKKVE | |
| | PKSCDKTHTCPPCPAPELLGGPSVFLFPPKPKDT | |
| | LMISRTPEVTCVVVDVSHEDPEVKFNWYVDGV | |
| | EVHNAKTKPREEQYNSTYRVVSVLTVLHQDW | |
| | LNGKEYKCKVSNKALPAPIEKTISKAKGQPREP | |
| | QVYTLPPSRDELTKNQVSLSCAVKGFYPSDIAV | |
| | EWESNGQPENNYKTTPPVLDSDGSFFLVSKLTV | |
| | DKSRWQQGNVFSCSVMHEALHNHYTQKSLSL | |
| | SPGK | |
| | LC: | 25 |
| | AIQLTQSPSSLSASVGDRVTITCRASQDISSALV | |
| | WYQQKPGKAPKLLIYDASSLESGVPSRFSGSES | |
| | GTDFTLTISSLQPEDFATYYCQQFNSYPLTFGG | |
| | GTKVEIKRTVAAPSVFIFPPSDEQLKSGTASVVC | |
| | LLNNFYPREAKVQWKVDNALQSGNSQESVTE | |
| | QDSKDSTYSLSSTLTLSKADYEKHKVYACEVT | |
| | HQGLSSPVTKSFNRGEC | |
| Panitumumab | HC: | 26 |
| hole | QVQLQESGPGLVKPSETLSLTCTVSGGSVSSGD | |
| | YYWTWIRQSPGKGLEWIGHIYYSGNTNYNPSL | |
| | KSRLTISIDTSKTQFSLKLSSVTAADTAIYYCVR | |
| | DRVTGAFDIWGQGTMVTVSSASTKGPSVFPLA | |
| | PCSRSTSESTAALGCLVKDYFPEPVTVSWNSGA | |
| | LTSGVHTFPAVLQSSGLYSLSSVVTVPSSSLGTQ | |
| | TYICNVNHKPSNTKVDKKVEPKSCDKTHTCPP | |
| | CPAPELLGGPSVFLFPPKPKDTLMISRTPEVTCV | |
| | VVDVSHEDPEVKFNWYVDGVEVHNAKTKPRE | |
| | EQYNSTYRVVSVLTVLHQDWLNGKEYKCKVS | |
| | NKALPAPIEKTISKAKGQPREPQVYTLPPSRDEL | |
| | TKNQVSLSCAVKGFYPSDIAVEWESNGQPENN | |
| | YKTTPPVLDSDGSFFLVSKLTVDKSRWQQGNV | |
| | FSCSVMHEALHNHYTQKSLSLSPGK | |
| | LC: | 27 |
| | DIQMTQSPSSLSASVGDRVTITCQASQDISNYLN | |
| | WYQQKPGKAPKLLIYDASNLETGVPSRFSGSGS | |
| | GTDFTFTISSLQPEDIATYFCQHFDHLPLAFGGG | |
| | TKVEIKRTVAAPSVFIFPPSDEQLKSGTASVVCL | |
| | LNNFYPREAKVQWKVDNALQSGNSQESVTEQ | |
| | DSKDSTYSLSSTLTLSKADYEKHKVYACEVTH | |
| | QGLSSPVTKSFNRGEC | |
| Necitumumab | HC: | 28 |
| hole | QVQLQESGPGLVKPSQTLSLTCTVSGGSISSGD | |
| | YYWSWIRQPPGKGLEWIGYIYYSGSTDYNPSL | |
| | KSRVTMSVDTSKNQFSLKVNSVTAADTAVYY | |
| | CARVSIFGVGTFDYWGQGTLVTVSSASTKGPS | |
| | VLPLAPSSKSTSGGTAALGCLVKDYFPEPVTVS | |
| | WNSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMISRT | |
| | PEVTCVVVDVSHEDPEVKFNWYVDGVEVHNA | |
| | KTKPREEQYNSTYRVVSVLTVLHQDWLNGKE | |
| | YKCKVSNKALPAPIEKTISKAKGQPREPQVYTL | |
| | PPSRDELTKNQVSLSCAVKGFYPSDIAVEWESN | |
| | GQPENNYKTTPPVLDSDGSFFLVSKLTVDKSR | |
| | WQQGNVFSCSVMHEALHNHYTQKSLSLSPGK | |
| | LC: | 29 |
| | EIVMTQSPATLSLSPGERATLSCRASQSVSSYLA | |
| | WYQQKPGQAPRLLIYDASNRATGIPARFSGSGS | |
| | GTDFTLTISSLEPEDFAVYYCHQYGSTPLTFGG | |
| | GTKAEIKRTVAAPSVFIFPPSDEQLKSGTASVVC | |
| | LLNNFYPREAKVQWKVDNALQSGNSQESVTE | |
| | QDSKDSTYSLSSTLTLSKADYEKHKVYACEVT | |
| | HQGLSSPVTKSFNRGEC | |
| Matuzumab | HC: | 30 |
| hole | QVQLVQSGAEVKKPGASVKVSCKASGYTFTSH | |
| | WMHWVRQAPGQGLEWIGEFNPSNGRTNYNEK | |
| | FKSKATMTVDTSTNTAYMELSSLRSEDTAVYY | |
| | CASRDYDYDGRYFDYWGQGTLVTVSSASTKG | |
| | PSVFPLAPSSKSTSGGTAALGCLVKDYFPEPVT | |
| | VSWNSGALTSGVHTFPAVLQSSGLYSLSSVVT | |
| | VPSSSLGTQTYICNVNHKPSNTKVDKKVEPKSC | |
| | DKTHTCPPCPAPELLGGPSVFLFPPKPKDTLMIS | |
| | RTPEVTCVVVDVSHEDPEVKFNWYVDGVEVH | |
| | NAKTKPREEQYNSTYRVVSVLTVLHQDWLNG | |
| | KEYKCKVSNKALPAPIEKTISKAKGQPREPQVY | |
| | TLPPSRDELTKNQVSLSCAVKGFYPSDIAVEWE | |
| | SNGQPENNYKTTPPVLDSDGSFFLVSKLTVDKS | |
| | RWQQGNVFSCSVMHEALHNHYTQKSLSLSPG | |
| | K | |
| | LC: | 31 |
| | DIQMTQSPSSLSASVGDRVTITCSASSSVTYMY | |
| | WYQQKPGKAPKLLIYDTSNLASGVPSRFSGSGS | |
| | GTDYTFTISSLQPEDIATYYCQQWSSHIFTFGQG | |
| | TKVEIKRTVAAPSVFIFPPSDEQLKSGTASVVCL | |
| | LNNFYPREAKVQWKVDNALQSGNSQESVTEQ | |
| | DSKDSTYSLSSTLTLSKADYEKHKVYACEVTH | |
| | QGLSSPVTKSFNRGEC | |
| Trastuzumab | HC: | 32 |
| knob (anti- | EVQLVESGGGLVQPGGSLRLSCAASGFNIKDTY | |
| HER2) | IHWVRQAPGKGLEWVARIYPTNGYTRYADSV | |
| | KGRFTISADTSKNTAYLQMNSLRAEDTAVYYC | |
| | SRWGGDGFYAMDYWGQGTLVTVSSASTKGPS | |
| | VFPLAPSSKSTSGGTAALGCLVKDYFPEPVTVS | |
| | WNSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMISRT | |
| | PEVTCVVVDVSHEDPEVKFNWYVDGVEVHNA | |
| | KTKPREEQYNSTYRVVSVLTVLHQDWLNGKE | |
| | YKCKVSNKALPAPIEKTISKAKGQPREPQVYTL | |
| | PPSRDELTKNQVSLWCLVKGFYPSDIAVEWES | |
| | NGQPENNYKTTPPVLDSDGSFFLYSKLTVDKSR | |
| | WQQGNVFSCSVMHEALHNHYTQKSLSLSPGK | |
| | GGSHHHHHH | |
| | LC: | 33 |
| | DIQMTQSPSSLSASVGDRVTITCRASQDVNTAV | |
| | AWYQQKPGKAPKLLIYSASFLYSGVPSRFSGSR | |
| | SGTDFTLTISSLQPEDFATYYCQQHYTTPPTFGQ | |
| | GTKLEIKRTVAAPSVFIFPPSDEQLKSGTASVVC | |
| | LLNNFYPREAKVQWKVDNALQSGNSQESVTE | |
| | QDSKDSTYSLSSTLTLSKADYEKHKVYACEVT | |
| | HQGLSSPVTKSFNRGEC | |
| Polatuzumab | HC: | 34 |
| knob (anti- | EVQLVESGGGLVQPGGSLRLSCAASGYTFSSY | |
| CD79B) | WIEWVRQAPGKGLEWIGEILPGGGDTNYNEIF | |
| | KGRATFSADTSKNTAYLQMNSLRAEDTAVYY | |
| | CTRRVPIRLDYWGQGTLVTVSSASTKGPSVFPL | |
| | APSSKSTSGGTAALGCLVKDYFPEPVTVSWNS | |
| | GALTSGVHTFPAVLQSSGLYSLSSVVTVPSSSL | |
| | GTQTYICNVNHKPSNTKVDKKVEPKSCDKTHT | |
| | CPPCPAPELLGGPSVFLFPPKPKDTLMISRTPEV | |
| | TCVVVDVSHEDPEVKFNWYVDGVEVHNAKTK | |
| | PREEQYNSTYRVVSVLTVLHQDWLNGKEYKC | |
| | KVSNKALPAPIEKTISKAKGQPREPQVYTLPPSR | |
| | DELTKNQVSLWCLVKGFYPSDIAVEWESNGQP | |
| | ENNYKTTPPVLDSDGSFFLYSKLTVDKSRWQQ | |
| | GNVFSCSVMHEALHNHYTQKSLSLSPGKGGSH | |
| | HHHHH | |
| | LC: | 35 |
| | DIQLTQSPSSLSASVGDRVTITCKASQSVDYEG | |
| | DSFLNWYQQKPGKAPKLLIYAASNLESGVPSRF | |
| | SGSGSGTDFTLTISSLQPEDFATYYCQQSNEDPL | |
| | TFGQGTKVEIKRTVAAPSVFIFPPSDEQLKSGTA | |
| | SVVCLLNNFYPREAKVQWKVDNALQSGNSQE | |
| | SVTEQDSKDSTYSLSSTLTLSKADYEKHKVYA | |
| | CEVTHQGLSSPVTKSFNRGEC | |
| Belantamab | HC: | 36 |
| knob (anti- | QVQLVQSGAEVKKPGSSVKVSCKASGGTFSNY | |
| BCMA) | WMHWVRQAPGQGLEWMGATYRGHSDTYYN | |
| | QKFKGRVTITADKSTSTAYMELSSLRSEDTAVY | |
| | YCARGAIYDGYDVLDNWGQGTLVTVSSASTK | |
| | GPSVFPLAPSSKSTSGGTAALGCLVKDYFPEPV | |
| | TVSWNSGALTSGVHTFPAVLQSSGLYSLSSVVT | |
| | VPSSSLGTQTYICNVNHKPSNTKVDKKVEPKSC | |
| | DKTHTCPPCPAPELLGGPSVFLFPPKPKDTLMIS | |
| | RTPEVTCVVVDVSHEDPEVKFNWYVDGVEVH | |
| | NAKTKPREEQYNSTYRVVSVLTVLHQDWLNG | |
| | KEYKCKVSNKALPAPIEKTISKAKGQPREPQVY | |
| | TLPPSRDELTKNQVSLWCLVKGFYPSDIAVEW | |
| | ESNGQPENNYKTTPPVLDSDGSFFLYSKLTVDK | |
| | SRWQQGNVFSCSVMHEALHNHYTQKSLSLSPG | |
| | KGGSHHHHHH | |
| | LC: | 37 |
| | DIQMTQSPSSLSASVGDRVTITCSASQDISNYLN | |
| | WYQQKPGKAPKLLIYYTSNLHSGVPS | |
| | RFSGSGSGTDFTLTISSLQPEDFATYYCQQYRK | |
| | LPWTFGQGTKLEIKRTVAAPSVFIFPPSDEQLKS | |
| | GTASVVCLLNNFYPREAKVQWKVDNALQSGN | |
| | SQESVTEQDSKDSTYSLSSTLTLSKADYEKHKV | |
| | YACEVTHQGLSSPVTKSFNRGEC | |
| Zalutumumab | HC: | 38 |
| knob (anti- | QVQLVESGGGVVQPGRSLRLSCAASGFTFSTY | |
| EGFR) | GMHWVRQAPGKGLEWVAVIWDDGSYKYYGD | |
| | SVKGRFTISRDNSKNTLYLQMNSLRAEDTAVY | |
| | YCARDGITMVRGVMKDYFDYWGQGTLVTVSS | |
| | ASTKGPSVFPLAPSSKSTSGGTAALGCLVKDYF | |
| | PEPVTVSWNSGALTSGVHTFPAVLQSSGLYSLS | |
| | SVVTVPSSSLGTQTYICNVNHKPSNTKVDKRVE | |
| | PKSCDKTHTCPPCPAPELLGGPSVFLFPPKPKDT | |
| | LMISRTPEVTCVVVDVSHEDPEVKFNWYVDGV | |
| | EVHNAKTKPREEQYNSTYRVVSVLTVLHQDW | |
| | LNGKEYKCKVSNKALPAPIEKTISKAKGQPREP | |
| | QVYTLPPSRDELTKNQVSLWCLVKGFYPSDIA | |
| | VEWESNGQPENNYKTTPPVLDSDGSFFLYSKLT | |
| | VDKSRWQQGNVFSCSVMHEALHNHYTQKSLS | |
| | LSPGKGGSHHHHHH | |
| | LC: | 39 |
| | AIQLTQSPSSLSASVGDRVTITCRASQDISSALV | |
| | WYQQKPGKAPKLLIYDASSLESGVPSRFSGSES | |
| | GTDFTLTISSLQPEDFATYYCQQFNSYPLTFGG | |
| | GTKVEIKRTVAAPSVFIFPPSDEQLKSGTASVVC | |
| | LLNNFYPREAKVQWKVDNALQSGNSQESVTE | |
| | QDSKDSTYSLSSTLTLSKADYEKHKVYACEVT | |
| | HQGLSSPVTKSFNRGEC | |
| Gemtuzumab | HC: | 40 |
| knob (anti- | EVQLVQSGAEVKKPGSSVKVSCKASGYTITDS | |
| CD33) | NIHWVRQAPGQSLEWIGYIYPYNGGTDYNQKF | |
| | KNRATLTVDNPTNTAYMELSSLRSEDTAFYYC | |
| | VNGNPWLAYWGQGTLVTVSSASTKGPSVFPLA | |
| | PCSRSTSESTAALGCLVKDYFPEPVTVSWNSGA | |
| | LTSGVHTFPAVLQSSGLYSLSSVVTVPSSSLGTK | |
| | TYTCNVDHKPSNTKVDKRVEPKSCDKTHTCPP | |
| | CPAPELLGGPSVFLFPPKPKDTLMISRTPEVTCV | |
| | VVDVSHEDPEVKFNWYVDGVEVHNAKTKPRE | |
| | EQYNSTYRVVSVLTVLHQDWLNGKEYKCKVS | |
| | NKALPAPIEKTISKAKGQPREPQVYTLPPSRDEL | |
| | TKNQVSLWCLVKGFYPSDIAVEWESNGQPENN | |
| | YKTTPPVLDSDGSFFLYSKLTVDKSRWQQGNV | |
| | FSCSVMHEALHNHYTQKSLSLSPGKGGSHHHH | |
| | HH | |
| | LC: | 41 |
| | DIQLTQSPSTLSASVGDRVTITCRASESLDNYGI | |
| | RFLTWFQQKPGKAPKLLMYAASNQGSGVPSRF | |
| | SGSGSGTEFTLTISSLQPDDFATYYCQQTKEVP | |
| | WSFGQGTKVEVKRTVAAPSVFIFPPSDEQLKSG | |
| | TASVVCLLNNFYPREAKVQWKVDNALQSGNS | |
| | QESVTEQDSKDSTYSLSSTLTLSKADYEKHKVY | |
| | ACEVTHQGLSSPVTKSFNRGEC | |
| Inotuzumab | HC: | 42 |
| knob (anti- | EVQLVQSGAEVKKPGASVKVSCKASGYRFTNY | |
| CD22) | WIHWVRQAPGQGLEWIGGINPGNNYATYRRK | |
| | FQGRVTMTADTSTSTVYMELSSLRSEDTAVYY | |
| | CTREGYGNYGAWFAYWGQGTLVTVSSASTKG | |
| | PSVFPLAPCSRSTSESTAALGCLVKDYFPEPVTV | |
| | SWNSGALTSGVHTFPAVLQSSGLYSLSSVVTVP | |
| | SSSLGTKTYTCNVDHKPSNTKVDKRVEPKSCD | |
| | KTHTCPPCPAPELLGGPSVFLFPPKPKDTLMISR | |
| | TPEVTCVVVDVSHEDPEVKFNWYVDGVEVHN | |
| | AKTKPREEQYNSTYRVVSVLTVLHQDWLNGK | |
| | EYKCKVSNKALPAPIEKTISKAKGQPREPQVYT | |
| | LPPSRDELTKNQVSLWCLVKGFYPSDIAVEWES | |
| | NGQPENNYKTTPPVLDSDGSFFLYSKLTVDKSR | |
| | WQQGNVFSCSVMHEALHNHYTQKSLSLSPGK | |
| | GGSHHHHHH | |
| | LC: | 43 |
| | DVQVTQSPSSLSASVGDRVTITCRSSQSLANSY | |
| | GNTFLSWYLHKPGKAPQLLIYGISNRFSGVPDR | |
| | FSGSGSGTDFTLTISSLQPEDFATYYCLQGTHQP | |
| | YTFGQGTKVEIKRTVAAPSVFIFPPSDEQLKSGT | |
| | ASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| Loncastuximab | HC: | 44 |
| knob (anti- | QVQLVQPGAEVVKPGASVKLSCKTSGYTFTSN | |
| CD19) | WMHWVKQAPGQGLEWIGEIDPSDSYTNYNQN | |
| | FQGKAKLTVDKSTSTAYMEVSSLRSDDTAVYY | |
| | CARGSNPYYYAMDYWGQGTSVTVSSASTKGP | |
| | SVFPLAPSSKSTSGGTAALGCLVKDYFPEPVTV | |
| | SWNSGALTSGVHTFPAVLQSSGLYSLSSVVTVP | |
| | SSSLGTQTYICNVNHKPSNTKVDKKVEPKSCD | |
| | KTHTCPPCPAPELLGGPSVFLFPPKPKDTLMISR | |
| | TPEVTCVVVDVSHEDPEVKFNWYVDGVEVHN | |
| | AKTKPREEQYNSTYRVVSVLTVLHQDWLNGK | |
| | EYKCKVSNKALPAPIEKTISKAKGQPREPQVYT | |
| | LPPSRDELTKNQVSLWCLVKGFYPSDIAVEWES | |
| | NGQPENNYKTTPPVLDSDGSFFLYSKLTVDKSR | |
| | WQQGNVFSCSVMHEALHNHYTQKSLSLSPGK | |
| | GGSHHHHHH | |
| | LC: | 45 |
| | EIVLTQSPAIMSASPGERVTMTCSASSGVNYMH | |
| | WYQQKPGTSPRRWIYDTSKLASGVPARFSGSG | |
| | SGTSYSLTISSMEPEDAATYYCHQRGSYTFGGG | |
| | TKLEIKRTVAAPSVFIFPPSDEQLKSGTASVVCL | |
| | LNNFYPREAKVQWKVDNALQSGNSQESVTEQ | |
| | DSKDSTYSLSSTLTLSKADYEKHKVYACEVTH | |
| | QGLSSPVTKSFNRGEC | |
| Sacituzumab | HC: | 46 |
| knob (anti- | QVQLQQSGSELKKPGASVKVSCKASGYTFTNY | |
| TROP2) | GMNWVKQAPGQGLKWMGWINTYTGEPTYTD | |
| | DFKGRFAFSLDTSVSTAYLQISSLKADDTAVYF | |
| | CARGGFGSSYWYFDVWGQGSLVTVSSASTKG | |
| | PSVFPLAPSSKSTSGGTAALGCLVKDYFPEPVT | |
| | VSWNSGALTSGVHTFPAVLQSSGLYSLSSVVT | |
| | VPSSSLGTQTYICNVNHKPSNTKVDKRVEPKSC | |
| | DKTHTCPPCPAPELLGGPSVFLFPPKPKDTLMIS | |
| | RTPEVTCVVVDVSHEDPEVKFNWYVDGVEVH | |
| | NAKTKPREEQYNSTYRVVSVLTVLHQDWLNG | |
| | KEYKCKVSNKALPAPIEKTISKAKGQPREPQVY | |
| | TLPPSRDELTKNQVSLWCLVKGFYPSDIAVEW | |
| | ESNGQPENNYKTTPPVLDSDGSFFLYSKLTVDK | |
| | SRWQQGNVFSCSVMHEALHNHYTQKSLSLSPG | |
| | KGGSHHHHHH | |
| | LC: | 47 |
| | DIQLTQSPSSLSASVGDRVSITCKASQDVSIAVA | |
| | WYQQKPGKAPKLLIYSASYRYTGVPDRFSGSG | |
| | SGTDFTLTISSLQPEDFAVYYCQQHYITPLTFGA | |
| | GTKVEIKRTVAAPSVFIFPPSDEQLKSGTASVVC | |
| | LLNNFYPREAKVQWKVDNALQSGNSQESVTE | |
| | QDSKDSTYSLSSTLTLSKADYEKHKVYACEVT | |
| | HQGLSSPVTKSFNRGEC | |
| Omburtamab | HC: | 48 |
| knob (anti-B7- | QVQLQQSGAELVKPGASVKLSCKASGYTFTNY | |
| H3) | DINWVRQRPEQGLEWIGWIFPGDGSTQYNEKF | |
| | KGKATLTTDTSSSTAYMQLSRLTSEDSAVYFC | |
| | ARQTTATWFAYWGQGTLVTVSAAKTTPPSVY | |
| | PLAPGSAAQTNSMVTLGCLVKGYFPEPVTVTW | |
| | NSGSLSSGVHTFPAVLQSDLYTLSSSVTVPSST | |
| | WPSETVTCNVAHPASSTKVDKKIVEPKSCDKT | |
| | HTCPPCPAPELLGGPSVFLFPPKPKDTLMISRTP | |
| | EVTCVVVDVSHEDPEVKFNWYVDGVEVHNAK | |
| | TKPREEQYNSTYRVVSVLTVLHQDWLNGKEY | |
| | KCKVSNKALPAPIEKTISKAKGQPREPQVYTLP | |
| | PSRDELTKNQVSLWCLVKGFYPSDIAVEWESN | |
| | GQPENNYKTTPPVLDSDGSFFLYSKLTVDKSR | |
| | WQQGNVFSCSVMHEALHNHYTQKSLSLSPGK | |
| | GGSHHHHHH | |
| | LC: | 49 |
| | DIVMTQSPATLSVTPGDRVSLSCRASQSISDYL | |
| | HWYQQKSHESPRLLIKYASQSISGIPSRFSGSGS | |
| | GSDFTLSINSVEPEDVGVYYCQNGHSFPLTFGA | |
| | GTKLELKRADAAPTVSIFPPSSEQLTSGGASVV | |
| | CFLNNFYPKDINVKWKIDGSERQNGVLNSWTD | |
| | QDSKDSTYSMSSTLTLTKDEYERHNSYTCEAT | |
| | HKTSTSPIVKSFNRNEC | |
| Tisotumab knob | HC: | 50 |
| (anti-Tissue | EVQLLESGGGLVQPGGSLRLSCAASGFTFSNYA | |
| factor) | MSWVRQAPGKGLEWVSSISGSGDYTYYTDSV | |
| | KGRFTISRDNSKNTLYLQMNSLRAEDTAVYYC | |
| | ARSPWGYYLDSWGQGTLVTVSSASTKGPSVFP | |
| | LAPSSKSTSGGTAALGCLVKDYFPEPVTVSWNS | |
| | GALTSGVHTFPAVLQSSGLYSLSSVVTVPSSSL | |
| | GTQTYICNVNHKPSNTKVDKRVEPKSCDKTHT | |
| | CPPCPAPELLGGPSVFLFPPKPKDTLMISRTPEV | |
| | TCVVVDVSHEDPEVKFNWYVDGVEVHNAKTK | |
| | PREEQYNSTYRVVSVLTVLHQDWLNGKEYKC | |
| | KVSNKALPAPIEKTISKAKGQPREPQVYTLPPSR | |
| | DELTKNQVSLWCLVKGFYPSDIAVEWESNGQP | |
| | ENNYKTTPPVLDSDGSFFLYSKLTVDKSRWQQ | |
| | GNVFSCSVMHEALHNHYTQKSLSLSPGKGGSH | |
| | HHHHH | |
| | LC: | 51 |
| | DIQMTQSPPSLSASAGDRVTITCRASQGISSRLA | |
| | WYQQKPEKAPKSLIYAASSLQSGVPSRFSGSGS | |
| | GTDFTLTISSLQPEDFATYYCQQYNSYPYTFGQ | |
| | GTKLEIKRTVAAPSVFIFPPSDEQLKSGTASVVC | |
| | LLNNFYPREAKVQWKVDNALQSGNSQESVTE | |
| | QDSKDSTYSLSSTLTLSKADYEKHKVYACEVT | |
| | HQGLSSPVTKSFNRGEC | |
| Farletuzumab | HC: | 52 |
| knob (anti- | EVQLVESGGGVVQPGRSLRLSCSASGFTFSGYG | |
| FOLR1) | LSWVRQAPGKGLEWVAMISSGGSYTYYADSV | |
| | KGRFAISRDNAKNTLFLQMDSLRPEDTGVYFC | |
| | ARHGDDPAWFAYWGQGTPVTVSSASTKGPSV | |
| | FPLAPSSKSTSGGTAALGCLVKDYFPEPVTVSW | |
| | NSGALTSGVHTFPAVLQSSGLYSLSSVVTVPSS | |
| | SLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAK | |
| | TKPREEQYNSTYRVVSVLTVLHQDWLNGKEY | |
| | KCKVSNKALPAPIEKTISKAKGQPREPQVYTLP | |
| | PSRDELTKNQVSLWCLVKGFYPSDIAVEWESN | |
| | GQPENNYKTTPPVLDSDGSFFLYSKLTVDKSR | |
| | WQQGNVFSCSVMHEALHNHYTQKSLSLSPGK | |
| | GGSHHHHHH | |
| | LC: | 53 |
| | DIQLTQSPSSLSASVGDRVTITCSVSSSISSNNLH | |
| | WYQQKPGKAPKPWIYGTSNLASGVPSRFSGSG | |
| | SGTDYTFTISSLQPEDIATYYCQQWSSYPYMYT | |
| | FGQGTKVEIKRTVAAPSVFIFPPSDEQLKSGTAS | |
| | VVCLLNNFYPREAKVQWKVDNALQSGNSQES | |
| | VTEQDSKDSTYSLSSTLTLSKADYEKHKVYAC | |
| | EVTHQGLSSPVTKSFNRGEC | |
| Apamistamab | HC: | 54 |
| knob (anti- | EVKLLESGGGLVQPGGSLKLSCAASGFDFSRY | |
| CD45) | WMSWVRQAPGKGLEWIGEINPTSSTINFTPSLK | |
| | DKVFISRDNAKNTLYLQMSKVRSEDTALYYCA | |
| | RGNYYRYGDAMDYWGQGTSVTVSSAKTTPPS | |
| | VYPLAPGSAAQTNSMVTLGCLVKGYFPEPVTV | |
| | TWNSGSLSSGVHTFPAVLQSDLYTLSSSVTVPS | |
| | STWPSETVTCNVAHPASSTKVDKKIVEPKSCDK | |
| | THTCPPCPAPELLGGPSVFLFPPKPKDTLMISRT | |
| | PEVTCVVVDVSHEDPEVKFNWYVDGVEVHNA | |
| | KTKPREEQYNSTYRVVSVLTVLHQDWLNGKE | |
| | YKCKVSNKALPAPIEKTISKAKGQPREPQVYTL | |
| | PPSRDELTKNQVSLWCLVKGFYPSDIAVEWES | |
| | NGQPENNYKTTPPVLDSDGSFFLYSKLTVDKSR | |
| | WQQGNVFSCSVMHEALHNHYTQKSLSLSPGK | |
| | GGSHHHHHH | |
| | LC | 55 |
| | DIALTQSPASLAVSLGQRATISCRASKSVSTSGY | |
| | SYLHWYQQKPGQPPKLLIYLASNLESGVPARFS | |
| | GSGSGTDFTLNIHPVEEEDAATYYCQHSRELPF | |
| | TFGSGTKLEIKRADAAPTVSIFPPSSEQLTSGGA | |
| | SVVCFLNNFYPKDINVKWKIDGSERQNGVLNS | |
| | WTDQDSKDSTYSMSSTLTLTKDEYERHNSYTC | |
| | EATHKTSTSPIVKSFNRNEC | |
| Serotransferrin | Knob: | 56 |
| knob (anti- | VPDKTVRWCAVSEHEATKCQSFRDHMKSVIPS | |
| TFRC) | DGPSVACVKKASYLDCIRAIAANEADAVTLDA | |
| | GLVYDAYLAPNNLKPVVAEFYGSKEDPQTFYY | |
| | AVAVVKKDSGFQMNQLRGKKSCHTGLGRSAG | |
| | WNIPIGLLYCDLPEPRKPLEKAVANFFSGSCAP | |
| | CADGTDFPQLCQLCPGCGCSTLNQYFGYSGAF | |
| | KCLKDGAGDVAFVKHSTIFENLANKADRDQYE | |
| | LLCLDNTRKPVDEYKDCHLAQVPSHTVVARS | |
| | MGGKEDLIWELLNQAQEHFGKDKSKEFQLFSS | |
| | PHGKDLLFKDSAHGFLKVPPRMDAKMYLGYE | |
| | YVTAIRNLREGTCPEAPTDECKPVKWCALSHH | |
| | ERLKCDEWSVNSVGKIECVSAETTEDCIAKIMN | |
| | GEADAMSLDGGFVYIAGKCGLVPVLAENYNK | |
| | SDNCEDTPEAGYFAIAVVKKSASDLTWDNLKG | |
| | KKSCHTAVGRTAGWNIPMGLLYNKINHCRFDE | |
| | FFSEGCAPGSKKDSSLCKLCMGSGLNLCEPNN | |
| | KEGYYGYTGAFRCLVEKGDVAFVKHQTVPQN | |
| | TGGKNPDPWAKNLNEKDYELLCLDGTRKPVE | |
| | EYANCHLARAPNHAVVTRKDKEACVHKILRQ | |
| | QQHLFGSNVTDCSGNFCLFRSETKDLLFRDDTV | |
| | CLAKLHDRNTYEKYLGEEYVKAVGNLRKCSTS | |
| | SLLEACTFRRPEPKSCDKTHTCPPCPAPELLGGP | |
| | SVFLFPPKPKDTLMISRTPEVTCVVVDVSHEDP | |
| | EVKFNWYVDGVEVHNAKTKPREEQYNSTYRV | |
| | VSVLTVLHQDWLNGKEYKCKVSNKALPAPIEK | |
| | TISKAKGQPREPQVYTLPPSRDELTKNQVSLWC | |
| | LVKGFYPSDIAVEWESNGQPENNYKTTPPVLDS | |
| | DGSFFLYSKLTVDKSRWQQGNVFSCSVMHEAL | |
| | HNHYTQKSLSLSPGKGGSHHHHHH | |

In some embodiments, the first binding domain binds to an epitope of the endogenous internalizing receptor on the target cell, wherein the epitope comprises at least 70% sequence identity to an epitope to which Enfortumab binds. In some embodiments, the first binding domain binds to an epitope of the endogenous internalizing receptor on the target cell, wherein the epitope comprises at least 8000 sequence identity to an epitope to which Enfortumab binds. In some cases, the first binding domain binds to an epitope of the endogenous internalizing receptor on the target cell, wherein the epitope comprises at least 90% sequence identity to the epitope to which Enfortumab binds. In some embodiments, the first binding domain binds to an epitope of the endogenous internalizing receptor on the target cell, wherein the epitope comprises at least 95% sequence identity to an epitope to which Enfortumab binds.

In some embodiments, the first binding domain binds to an epitope of the endogenous internalizing receptor on the target cell that does not include any of the amino acids from the epitope to which Enfortumab binds. In some embodiments, the first binding domain binds to an epitope of the endogenous internalizing receptor on the target cell that includes one, two, three, four, five, or six of the amino acids from the epitope to which Enfortumab binds. In some embodiments, the first binding domain binds to an epitope of the endogenous internalizing receptor on the target cell that includes one or more of the amino acids from the epitope to which Enfortumab binds. In some embodiments, the first binding domain binds to an epitope of the endogenous internalizing receptor on the target cell that includes two or more of the amino acids from the epitope to which Enfortumab binds. In some embodiments, the first binding domain binds to an epitope of the endogenous internalizing receptor on the target cell that includes three or more of the amino acids from the epitope to which Enfortumab binds. In some embodiments, the first binding domain binds to an epitope of the endogenous internalizing receptor on the target cell that includes four or more of the amino acids from the epitope to which Enfortumab binds.

The bispecific binding agents provided herein further include a second binding domain that can specifically bind to a target protein. The target protein can be a soluble target protein and a membrane-associated target protein. In some embodiments, the second binding domain of the bispecific binding agents provided herein can bind to an extracellular epitope of a membrane-associated target protein. The binding of the second binding domain to the membrane-associated target protein can result in the internalization of a target cell expressing the membrane-associated target protein.

In some embodiments, the target protein of the bispecific binding agents provided herein can be an immune checkpoint protein. Immune checkpoint proteins are known in the field, and generally refers to proteins that serve as checkpoints produced by some types of immune system cells, such as T cells, and some cancer cells. Some non-limiting examples of immune checkpoint proteins include PD-L1, PD-1, CTLA-4, B7-H3, B7-H4, BTLA, KIR, LAG3, NKG2D, TIM-3, VISTA, SIGLEC7, and SIGLEC15.

In some embodiments, the target protein of the bispecific binding agents provided herein can be a cancer antigen. In some embodiments, cancer antigens are proteins that are expressed on the surface of certain cancer cells. In other embodiments, cancer antigens are shed by the cancer cells and can be detected in blood and sometimes other body fluids. Thus, cancer antigens can include both cell membrane-associated target proteins and soluble target proteins. Some non-limiting examples of the cancer antigens include EGFR, CDCP1, CD38, IGF-1R, and MMP14.

In some embodiments, the target protein of the bispecific binding agents provided herein can be an immunomodulatory protein. Immunomodulatory proteins can refer to any proteins that have immunomodulatory activities. For instance, an immunomodulatory protein can have the signaling activity upon a certain stimulation that leads to either increased activity of immune cells (i.e., immune activation) or decreased activity of immune cells (i.e., immune suppression). Some immunomodulatory proteins may also have immune checkpoint activities. Thus, in some instances, immunomodulatory proteins could overlap with immune checkpoint proteins. Some non-limiting examples of the immuno-modulatory proteins include PD-L1, PD-1, CTLA-4, B7-H3, B7-H4, LAG3, NKG2D, TIM-3, VISTA, CD39, CD73 (NT5E), A2AR, SIGLEC7, and SIGLEC15.

In some embodiments, the target protein can be an inflammation receptor. Some non-limiting exemplary inflammation receptors include TNFR, IL1R, IL2Ralpha, IL2Rbeta.

Other cancer antigens, immuno-modulatory proteins, inflammation receptors, and T cell marker are known in the field and are also encompassed by the present disclosure. In certain embodiments, some non-limiting examples of the target proteins include PD-L1, HER2, EGFR, PD-1, CTLA-4, A2AR, B7-H3, B7-H4, BTLA, KIR, LAG3, NKG2D, TIM-3, VISTA, LAG3, NKG2D, TIM, SIGLEC7, SIGLEC15, CD19, CD20, CDCP1, MMP14, and TROP2.

In some embodiments, the bispecific binding agent comprises a first binding domain that binds to HER2, CD30, CD79B, Nectin-4, BCMA, EGFR, CD33, CD20, CD22, CD19, TROP2, B7-H3, Tissue factor, FOLR1, CD45, or TFRC and a second binding domain that binds to a target protein selected from EGFR, CDCP1, CD38, IGF-1R, and MMP14. In some embodiments, the bispecific binding agent comprises a first binding domain that binds to Nectin-4 and a second binding domain that binds to CDCP1. In some embodiments, the bispecific binding agent comprises a first binding domain that binds to Nectin-4 and a second binding domain that binds to PD-L1. In some embodiments, the bispecific binding agent comprises a first binding domain that binds to Nectin-4 and a second binding domain that binds to HER2. In some embodiments, the bispecific binding agent comprises a first binding domain that binds to Nectin-4 and a second binding domain that binds to EGFR. In certain embodiments, the bispecific binding agent described herein is a bispecific antibody.

Without being bound by theory, in the cases where the target protein is a membrane-associated target protein, the target cell needs to express both the membrane-associated target protein and the internalizing receptor. For instance, a bispecific binding agent provided herein comprises (1) a first binding domain which specifically binds to Nectin-4, and (2) a second binding domain which includes a Fab targeting CDCP1. In this case, the target cell needs to express (1) Nectin-4 and (2) CDCP1.

In some embodiments, the bispecific binding agent comprising a first binding domain which specifically binds to Nectin-4 and a second binding domain which specifically binds to CDCP1 contacts a target cancer cell. In some embodiments, the bispecific binding agent comprising a first binding domain which specifically binds to Nectin-4 and a second binding domain which specifically binds to CDCP1 contacts a target bladder cancer cell. In some embodiments, the bispecific binding agent comprising a first binding domain which specifically binds to Nectin-4 and a second binding domain which specifically binds to CDCP1 contacts a target cancer cell and decreases expression of CDCP1 on the cancer cell by at least 40%, at least 50%, at least 55%, at least 60%, at least 65%, at least 70%, at least 75%, or at least 80%. In some embodiments, the bispecific binding agent comprising a first binding domain which specifically binds to Nectin-4 and a second binding domain which specifically binds to CDCP1 contacts a target cancer cell and decreases expression of CDCP1 on the cancer cell by about 40%-80%, about 50%-80%, about 60%-80%, about 70%-80%, about 40%-70%, about 50%-70%, about 60%-70%, about 40%-60%, or about 50%-60%. In some embodiments, expression of CDCP1 on a target cell is determined relative to expression of CDCP1 on a control cancer cell not contacted with the binding agent.

As mentioned above, in some embodiments, the bispecific binding agents of the present disclosure can specifically bind to an extracellular epitope of a membrane-associated target protein, and such binding can result in the membrane-associated target protein bound to the bispecific binding agent. Thus, one skilled in the art would appreciate that any cell expressing a target protein could be a target cell for the purpose of the present disclosure. For example, the target cells encompassed by the present can be a neoplastic cell. A neoplasm is an abnormal growth of cells. Neoplastic cells are cells that are undergoing or have undergone an abnormal growth. In some instances, these abnormally growing cells can cause tumor growth and can be both benign and malignant.

Alternatively, the target cells encompassed by the present disclosure can be cancer cells. Some non-limiting examples of target cells include cancer cells, such as cells from breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer.

In other embodiments, the target cells can be immune cells. For instance, the immune cells can be monocytes, macrophages, lymphocytes (e.g., natural killer cells, T cells, and B cells), and monocytes.

The second binding domain of the bispecific binding agents provided herein can also bind to soluble target proteins. In certain embodiments, the soluble target proteins include soluble extracellular proteins. For example, the soluble target protein that can be targeted by the bispecific binding agents provided herein include an inflammatory cytokine, a growth factor (GF), a toxic enzyme, a target associated with metabolic diseases, a neuronal aggregate, or an autoantibody. These various soluble proteins are known in the art. In some embodiments, non-limiting examples of the inflammatory cytokine include lymphotoxin, interleukin-1 (IL-1), IL-2, IL-5, IL-6, IL-12, IL-13, IL-17, IL-18, IL-23, tumor necrosis factor alpha (TNF-α), interferon gamma (IFNγ), and granulocyte-macrophage colony stimulating factor (GM-CSF). In other embodiments, non-limiting examples of the growth factor comprises EGF, FGF, NGF, PDGF, VEGF, IGF, GMCSF, GCSF, TGF, RANK-L, erythropieitn, TPO, BMP, HGF, GDF, neurotrophins, MSF, SGF, GDF, and an isoform thereof. In some embodiments, non-limiting examples of the toxic enzyme comprises a protein arginine deiminase 1 (PAD1), PAD2, PAD3, PAD4, and PAD6, leucocidin, hemolysin, coagulase, treptokinase, hyaluronidase. In certain embodiments, the toxic enzyme comprises PAD2 or PAD4. In some embodiments, the target associated with a metabolic disease can be PCSK9, HRD1 T2DM, and MOGAT2. In other embodiments, non-limiting examples of the neuronal aggregate comprises Aβ, TTR, α-synuclein, TAO, and prion. In certain embodiments, the autoantibody comprises IgA, IgE, IgG, IgM, and IgD. Target proteins associated with the conditions described herein are known in the field and new targets are being discovered. All of the known and to be discovered targets are encompassed herein.

In some embodiments, once bound by the second binding domain, the target protein is internalized at least 5%, 10%, 20%, 30%, 40%, 50%, 60%, 70%, 80%, 90% greater than a control.

The bispecific binding agents of the present disclosure can generally take the form of a protein, glycoprotein, lipoprotein, phosphoprotein, and the like. Some bispecific binding agent of the disclosure take the form of bispecific antibodies or antibody derivatives. In some embodiments, the first binding domain and the second binding domain of the bispecific binding agent provided herein can each be independently selected from the group consisting of natural ligands or a fragment, derivative, or small molecule mimetic thereof, IgG, half antibodies, single-domain antibodies, nanobodies, Fabs, monospecific Fab2, Fc, scFv, minibodies, IgNAR, V-NAR, hcIgG, VHH domains, camelid antibodies, and peptibodies. In some embodiments, the first binding domain and the second binding domain of the bispecific binding agent provided herein together can form a bispecific antibody, a bispecific diabody, a bispecific Fab2, a bispecific camelid antibody, or a bispecific peptibody scFv-Fc, a bispecific IgG, and a knob and hole bispecific IgG, a Fc-Fab, and a knob and hole bispecific Fc-Fab.

For example, one can employ known techniques such as phage display to generate and select for small proteins having a binding domain similar to an antibody complementarity-determining region (CDR). In some embodiments, the first or second binding domain includes a scFv. In other embodiments, the first or second binding domain includes a Fab. The first binding domain can also be derived from a natural or synthetic ligand that specifically binds to at least one internalizing receptor. The second binding domain can be derived from any known or to be developed antigen binding agents, e.g., any therapeutic antibodies, that specifically binds to target protein, whether soluble or membrane-associated.

The binding domains can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., PD-L1) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. In some embodiments, binding affinity is measured by bio-layer interferometry. An antibody that selectively binds an antigen (such as PD-L1) when it is capable of binding that antigen with high affinity, without significantly binding other antigens.

Bispecific antibodies can be prepared by known methods. Embodiments of the disclosure include “knob-into-hole” bispecific antibodies, wherein the otherwise symmetric dimerization region of a bispecific binding agent is altered so that it is asymmetric. For example, a knob-into-hole bispecific IgG that is specific for antigens A and B can be altered so that the Fc portion of the A-binding chain has one or more protrusions (“knobs”), and the Fc portion of the B-binding chain has one or more hollows (“holes”), where the knobs and holes are arranged to interact. This reduces the homodimerization (A-A and B-B antibodies), and promotes the heterodimerization desired for a bispecific binding agent. See, e.g., Y. Xu et al., mAbs (2015) 7(1):231-42. In some embodiments, the bispecific binding agent has a knob-into-hole design. In some embodiments, the “knob” comprises a T336W alteration of the CH3 domain, i.e., the threonine at position 336 is replaced by a tryptophan. In some embodiments, the “hole” comprises one or a combination of T366S, L368A, and Y407V. In some embodiments, the “hole” comprises T366S, L368A, and Y407V. In some exemplary embodiments, the “knob” constant region comprises a sequence set forth in SEQ ID NO: 57, 61, 65, 69, 73 or a portion of any one thereof. In some embodiments, the heavy chain Fc “knob” constant region has a histidine tag. In some exemplary embodiments, the heavy chain Fc “hole” constant region comprises SEQ ID NO: 59, 63, 67, 71 or a portion of any one thereof. In certain embodiments, an exemplary CH2-CH3 domain sequence of a Knob construct comprises a N297G. In other embodiments, an exemplary CH2-CH3 domain sequence of a Hole construct comprises N297G.

In other embodiments, the “knob” and the “hole” constant regions comprise sequences that are about 70%, 75%, 80%, 85%, 90%, 95%, 99% identical to the sequences provided herein. For example, see Table 2 for exemplary constructs and sequences.

In some embodiments, the first binding domain of the bispecific binding agent provided herein comprises an Fc-Fab. In some embodiments, the second binding domain comprises an Fc-Fab. In some embodiments, the second binding domain comprises an scFv. In some embodiments, the internalizing receptor to which the first binding domain binds to is referred to as a degrader, and the target protein of the second binding domain is referred to a victim.

Without being bound by theory, the present disclosure provides some exemplary bispecific binding agents that comprises binding agents to Nectin-4 in a Knob-Fc format as the first binding domain, and a Fab in a Hole-Fc format that specifically binds to various targets, including PD-L1, HER2, EGFR, and CDCP1, as the second binding domain. Table 3 below provides some exemplary designs and sequences of the bispecific binding agents of the present disclosure. Table 4 below provides a sequence for a binding agent against TROP2.

TABLE 3
Exemplary design and sequences of the bispecific binding agents.

| | | SEQ ID |
|---|---|---|
| Protein Name | Sequence | NO: |
| Enfortumab | Knob HC: | 57 |
| (Nectin-4)- | EVQLVESGGGLVQPGGSLRLSCAASGFTFSSYNMNWVR | |
| 4A06 | QAPGKGLEWVSYISSSSSTIYYADSVKGRFTISRDNAKNS | |
| (CDCP1) | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | 58 |
| | DIQMTQSPSSVSASVGDRVTITCRASQGISGWLAWYQQK | |
| | PGKAPKFLIYAASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 59 |
| | EISEVQLVESGGGLVQPGGSLRLSCAASGFNLSYYYIHW | |
| | VRQAPGKGLEWVASIYSSSSYTSYADSVKGRFTISADTS | |
| | KNTAYLQMNSLRAEDTAVYYCARAYYGFDYWGQGTL | |
| | VTVSSASTKGPSVFPLAPSSKSTSGGTAALGCLVKDYFPE | |
| | PVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLT | |
| | VLHQDWLNGKEYKCKVSNKALPAPIEKTISKAKGQPREP | |
| | QVYTLPPIRELMTSNQVSLSCAVKGFYPSDIAVEWESNG | |
| | QPENNYKTTPPVLDSDGSFFLVSKLTVDKSRWQQGNVF | |
| | SCSVMHEALHNHYTQKSLSLSPGK | |
| | Hole LC: | 60 |
| | DIQMTQSPSSLSASVGDRVTITCRASQSVSSAVAWYQQK | |
| | PGKAPKLLIYSASSLYSGVPSRFSGSRSGTDFTLTISSLQP | |
| | EDFATYYCQQSYYYYPITFGQGTKVEIKRTVAAPSVFIFP | |
| | PSDSQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGECGGSDYKDDDDK | |
| Enfortumab- | Knob HC: | |
| Tecentriq | EVQLVESGGGLVQPGGSLRLSCAASGFTFSSYNMNWVR | |
| (PD-L1) | QAPGKGLEWVSYISSSSSTIYYADSVKGRFTISRDNAKNS | |
| | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | |
| | DIQMTQSPSSVSASVGDRVTITCRASQGISGWLAWYQQK | |
| | PGKAPKFLIYAASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 63 |
| | EVQLVESGGGLVQPGGSLRLSCAASGFTFSDSWIHWVR | |
| | QAPGKGLEWVAWISPYGGSTYYADSVKGRFTISADTSK | |
| | NTAYLQMNSLRAEDTAVYYCARRHWPGGFDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPS | |
| | SSLGTQTYICNVNHKPSNTKVDKKVEPKSCEPKSCDKTH | |
| | TCPPCPAPELLGGPSVFLFPPKPKDTLMISRTPEVTCVVV | |
| | DVSHEDPEVKFNWYVDGVEVHNAKTKPREEQYGSTYR | |
| | VVSVLTVLHQDWLNGKEYKCKVSNKALPAPIEKTISKA | |
| | KGQPREPQVYTLPPSRDELTKNQVSLSCAVKGFYPSDIA | |
| | VEWESNGQPENNYKTTPPVLDSDGSFFLVSKLTVDKSR | |
| | WQQGNVFSCSVMHEALHNHYTQKSLSLSPGKGGSGAW | |
| | SHPQFEK | |
| | Hole LC: | 64 |
| | DIQMTQSPSSLSASVGDRVTITCRASQDVSTAVAWYQQK | |
| | PGKAPKLLIYSASFLYSGVPSRFSGSGSGTDFTLTISSLQP | |
| | EDFATYYCQQYLYHPATFGQGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| Enfortumab- | Knob HC: | 57 |
| Trastuzumab | EVQLVESGGGLVQPGGSLRLSCAASGFTFSSYNMNWVR | |
| (HER2) | QAPGKGLEWVSYISSSSSTIYYADSVKGRFTISRDNAKNS | |
| | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | 58 |
| | DIQMTQSPSSVSASVGDRVTITCRASQGISGWLAWYQQK | |
| | PGKAPKFLIYAASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 67 |
| | EVQLVESGGGLVQPGGSLRLSCAASGFNIKDTYIHWVRQ | |
| | APGKGLEWVARIYPTNGYTRYADSVKGRFTISADTSKNT | |
| | AYLQMNSLRAEDTAVYYCSRWGGDGFYAMDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPS | |
| | SSLGTQTYICNVNHKPSNTKVDKKVEPKSCDKTHTCPPC | |
| | PAPELLGGPSVFLFPPKPKDTLMISRTPEVTCVVVDVSHE | |
| | DPEVKFNWYVDGVEVHNAKTKPREEQYGSTYRVVSVL | |
| | TVLHQDWLNGKEYKCKVSNKALPAPIEKTISKAKGQPR | |
| | EPQVYTLPPSRDELTKNQVSLSCAVKGFYPSDIAVEWES | |
| | NGQPENNYKTTPPVLDSDGSFFLVSKLTVDKSRWQQGN | |
| | VFSCSVMHEALHNHYTQKSLSLSPGKGGSGAWSHPQFE | |
| | K | |
| | Hole LC: | 68 |
| | DIQMTQSPSSLSASVGDRVTITCRASQDVNTAVAWYQQ | |
| | KPGKAPKLLIYSASFLYSGVPSRFSGSRSGTDFTLTISSLQ | |
| | PEDFATYYCQQHYTTPPTFGQGTKLEIKRTVAAPSVFIFP | |
| | PSDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| Enfortumab- | Knob HC: | 57 |
| Cetuximab | EVQLVESGGGLVQPGGSLRLSCAASGFTFSSYNMNWVR | |
| (EGFR) | QAPGKGLEWVSYISSSSSTIYYADSVKGRFTISRDNAKNS | |
| | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | 58 |
| | DIQMTQSPSSVSASVGDRVTITCRASQGISGWLAWYQQK | |
| | PGKAPKFLIY AASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 71 |
| | QVQLKQSGPGLVQPSQSLSITCTVSGFSLTNYGVHWVRQ | |
| | SPGKGLEWLGVIWSGGNTDYNTPFTSRLSINKDNSKSQV | |
| | FFKMNSLQSNDTAIYYCARALTYYDYEFAYWGQGTLVT | |
| | VSAASTKGPSVFPLAPSSKSTSGGTAALGCLVKDYFPEP | |
| | VTVSWNSGALTSGVHTFPAVLQSSGLYSLSSVVTVPSSS | |
| | LGTQTYICNVNHKPSNTKVDKKVEPKSCDKTHTCPPCPA | |
| | PELLGGPSVFLFPPKPKDTLMISRTPEVTCVVVDVSHEDP | |
| | EVKFNWYVDGVEVHNAKTKPREEQYNSTYRVVSVLTV | |
| | LHQDWLNGKEYKCKVSNKALPAPIEKTISKAKGQPREPQ | |
| | VYTLPPSRDELTKNQVSLSCAVKGFYPSDIAVEWESNGQ | |
| | PENNYKTTPPVLDSDGSFFLVSKLTVDKSRWQQGNVFSC | |
| | SVMHEALHNHYTQKSLSLSPGK | |
| | Hole LC: | 72 |
| | DILLTQSPVILSVSPGERVSFSCRASQSIGTNIHWYQQRTN | |
| | GSPRLLIKYASESISGIPSRFSGSGSGTDFTLSINSVESEDIA | |
| | DYYCQQNNNWPTTFGAGTKLELKRTVAAPSVFIFPPSDE | |
| | QLKSGTASVVCLLNNFYPREAKVQWKVDNALQSGNSQ | |
| | ESVTEQDSKDSTYSLSSTLTLSKADYEKHKVYACEVTHQ | |
| | GLSSPVTKSFNRGEC | |

In some cases, the first binding domain binds to an epitope of the endogenous internalizing receptor on the target cell that does not include any of the amino acids from the epitope to which Enfortumab binds. In some embodiments, the first binding domain comprises a first binding domain variable heavy chain and a first binding domain variable light chain. In certain embodiments, the first binding domain variable heavy chain comprises at least 80% sequence identity to SEQ ID NO: 57. In some embodiments, the first binding domain variable heavy chain comprises at least 90% sequence identity to SEQ ID NO: 57. In some embodiments, the first binding domain variable heavy chain comprises SEQ ID NO: 57. In some embodiments, the first binding domain variable light chain comprises at least 80% sequence identity to SEQ ID NO: 58. In certain embodiments, the first binding domain variable light chain comprises at least 90% sequence identity to SEQ ID NO 58 In some embodiments, the first binding domain variable light chain comprises SEQ ID NO: 58.

In some cases, the second binding domain comprises a second binding domain variable heavy chain and a second binding domain variable light chain. In certain embodiments, the second binding domain variable heavy chain comprises at least 80% sequence identity to SEQ ID NO: 59, 63, 67, or 71. In some embodiments, the second binding domain variable heavy chain comprises at least 90% sequence identity to SEQ ID NO: 59, 63, 67, or 71. In certain embodiments, the second binding domain variable heavy chain comprises any of SEQ ID NOs: 59, 63, 67, or 71. In certain embodiments, the second binding domain variable light chain comprises at least 80% sequence identity to any one of SEQ ID NOs: 60, 64, 68, or 72. In some embodiments, the second binding domain variable light chain comprises at least 90% sequence identity to any one of SEQ ID NOs: 60, 64, 68, or 72. In certain embodiments, the second binding domain variable light chain comprises any one of SEQ ID NOs: 60, 64, 68, or 72.

TABLE 4
Agent for TROP2

| Protein | | SEQ ID |
|---|---|---|
| Name | Sequence | NO |
| Sacituzumab | HC: | 73 |
| knob | QVQLQQSGSELKKPGASVKVSCKASGYTFTNYGMNWVK | |
| (TROP2) | QAPGQGLKWMGWINTYTGEPTYTDDFKGRFAFSLDTSVS | |
| | TAYLQISSLKADDTAVYFCARGGFGSSYWYFDVWGQGSL | |
| | VTVSSASTKGPSVFPLAPSSKSTSGGTAALGCLVKDYFPEP | |
| | VTVSWNSGALTSGVHTFPAVLQSSGLYSLSSVVTVPSSSL | |
| | GTQTYICNVNHKPSNTKVDKRVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLWCLVKGFYPSDIAVEWESNGQPENNY | |
| | KTTPPVLDSDGSFFLYSKLTVDKSRWQQGNVFSCSVMHE | |
| | ALHNHYTQKSLSLSPGKGGSHHHHHH | |
| | LC: | 74 |
| | DIQLTQSPSSLSASVGDRVSITCKASQDVSIAVAWYQQKPG | |
| | KAPKLLIYSASYRYTGVPDRFSGSGSGTDFTLTISSLQPEDF | |
| | AVYYCQQHYITPLTFGAGTKVEIKRTVAAPSVFIFPPSDEQ | |
| | LKSGTASVVCLLNNFYPREAKVQWKVDNALQSGNSQESV | |
| | TEQDSKDSTYSLSSTLTLSKADYEKHKVYACEVTHQGLSS | |
| | PVTKSFNRGEC | |

### Nucleic Acid Molecules

In one aspect, some embodiments disclosed herein relate to nucleic acid molecules comprising nucleotide sequences encoding the bispecific binding agents of the disclosure, including expression cassettes, and expression vectors containing these nucleic acid molecules operably linked to heterologous nucleic acid sequences such as, for example, regulatory sequences which direct in vivo expression of the bispecific binding agents in a host cell. In some embodiments, the bispecific binding agent described herein is expressed from a single genetic construct.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell or tissue and/or into an individual. Thus, in some embodiments, an expression cassette of the disclosure comprises a nucleotide sequence encoding a bispecific binding agent operably linked to expression control elements sufficient to guide expression of the cassette in vivo. In some embodiments, the expression control element comprises a promoter and/or an enhancer and optionally, any or a combination of other nucleic acid sequences capable of effecting transcription and/or translation of the coding sequence.

In some embodiments, the nucleotide sequence is incorporated into an expression vector. Vectors generally comprise a recombinant polynucleotide construct designed for transfer between host cells, which may be used for the purpose of transformation, i.e., the introduction of heterologous DNA into a host cell. As such, in some embodiments, the vector can be a replicon, such as a plasmid, phage, or cosmid, into which another DNA segment may be inserted so as to bring about the replication of the inserted segment. Expression vectors further include a promoter operably linked to the recombinant polynucleotide, such that the recombinant polynucleotide is expressed in appropriate cells, under appropriate conditions. In some embodiments, the expression vector is an integrating vector, which can integrate into host nucleic acids.

In some embodiments, the expression vector is a viral vector, which further includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. Retroviral vectors contain structural and functional genetic elements, or portions thereof, that are primarily derived from a retrovirus. Lentiviral vectors are viral vectors or plasmids containing structural and functional genetic elements, or portions thereof, including LTRs that are primarily derived from a lentivirus.

The nucleic acid sequences can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the proteins disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the proteins disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, or bacteriophage, as a linear or circular, single-stranded or double-stranded, DNA or RNA polynucleotide, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any bispecific binding agent or engineered protein disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

The precise components of the expression system are not critical. For example, a bispecific binding agent as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally but encode the same gene product because the genetic code is degenerate. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., comprising either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a bispecific binding agent) can also be included. Those of ordinary skill in the art of molecular biology are familiar with routine procedures for isolating nucleic acid molecules. They can, for example, be generated by treatment of genomic DNA with restriction endonucleases, or by the polymerase chain reaction (PCR). In the event the nucleic acid molecule is a ribonucleic acid (RNA), transcripts can be produced, for example, by in vitro transcription.

### Recombinant Cells and Cell Cultures

The nucleic acid of the present disclosure can be introduced into a host cell, such as a human B lymphocyte, to produce a recombinant cell containing the nucleic acid molecule. Accordingly, some embodiments of the disclosure relate to methods for making recombinant cells, including the steps of: (a) providing a cell capable of protein expression and (b) contacting the provided cell with any of the recombinant nucleic acids described herein.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules are delivered to cells by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, or DNA-guided endonuclease genome editing NgAgo (Natronobacterium gregoryi Argonaute), or TALENs genome editing (transcription activator-like effector nucleases). In some embodiments, the nucleic acid molecule present in the recombinant host cell as a mini-circle expression vector for a stable or transient expression.

The nucleic acid molecules can be encapsulated in a viral capsid or a lipid nanoparticle. For example, introduction of nucleic acids into cells may be achieved by viral transduction. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also suitable for nucleic acid delivery and gene therapy via viral transduction. Lentiviral vectors offer several attractive properties as gene-delivery vehicles, including: (i) sustained gene delivery through stable vector integration into host genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells are genetically engineered (e.g., transduced, transformed, or transfected) with, for example, a vector comprising a nucleic acid sequence encoding a bispecific binding agent as described herein, either a virus-derived expression vector or a vector for homologous recombination further comprising nucleic acid sequences homologous to a portion of the genome of the host cell. Host cells can be either untransformed cells or cells that have already been transfected with one or more nucleic acid molecules.

In some embodiments, the recombinant cell is a prokaryotic cell or a eukaryotic cell. In some embodiments, the cell is transformed in vivo. In some embodiments, the cell is transformed ex vivo. In some embodiments, the cell is transformed in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a human subject.

In another aspect, provided herein are various cell cultures including at least one recombinant cell as disclosed herein, and a culture medium. Generally, the culture medium can be any one of suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Synthesis

Bispecific binding agents can be synthesized using the techniques of recombinant DNA and protein expression. For example, for the synthesis of DNA encoding a bispecific IgG of the disclosure, suitable DNA sequences encoding the constant domains of the heavy and light chains are widely available. Sequences encoding the selected variable domains are inserted by standard methods, and the resulting nucleic acids encoding full-length heavy and light chains are transduced into suitable host cells and expressed. Alternatively, the nucleic acids can be expressed in a cell-free expression system, which can provide more control over oxidation and reduction conditions, pH, folding, glycosylation, and the like.

In some embodiments, the bispecific binding agents can have two different complementary determining regions (CDRs), each specific for either the target protein or endogenous cell surface receptor. Thus, two different heavy chains and two different light chains are required. In other embodiments, the bispecific binding agents can have one or more CDRs specific for the target protein and a binding domain specific for the internalizing receptor. These may be expressed in the same host cell, and the resulting product will contain a mixture of homodimers and bispecific heterodimers. Homodimers can be separated from the bispecific antibodies by affinity purification (for example, first using beads coated with one antigen, then beads coated with the other antigen), reduced to monomers, and reassociated. Alternatively, one can employ a “knobs into holes” design, in which a dimerization region of a heavy chain constant region is altered so that the surface either protrudes (“knob”) from the surface (as compared to the wild type structure) or forms a cavity (“hole”) in such a way that the two modified surfaces are still capable of dimerizing. The knob heavy chain and its associated light chain are then expressed in one host cell, and the hole heavy chain and associated light chain are expressed in a different host cell, and the expressed proteins are combined. The asymmetry in the dimerization regions promotes the formation of heterodimers. To obtain dimerization, the two “monomers” (each consisting of a heavy chain and a light chain) are combined under reducing conditions at a moderately basic pH (e.g., about pH 8 to about pH 9) to promote disulfide bond formation between the appropriate heavy chain domains. See, e.g., U.S. Pat. No. 8,216,805 and EP 1870459A1, incorporated herein by reference.

Other methods can be used to promote heavy chain heterodimerization of the first and second polypeptide chains of bispecific antibodies. For example, in some embodiments, the heavy-chain heterodimerization of the first and second polypeptide chains of the engineered antibodies as disclosed herein can be achieved by a controlled Fab arm exchange method as described by F. L. Aran et al., Proc Natl Acad Sci USA (2013) 110(13):5145-50.

The dimerization process can result in exchange of the light chains between different heavy chain monomers. One method for avoiding this outcome is to replace the binding region of the antibody with a “single chain Fab”, e.g., wherein the light chain CDR is fused to the heavy chain CDR by a linking polypeptide. The Fab region of an IgG (or other antibody) may also be replaced with a scFv, nanobody, and the like.

The binding activity of the engineered antibodies of the disclosure can be assayed by any suitable method known in the art. For example, the binding activity of the engineered antibodies of the disclosure can be determined by, e.g., Scatchard analysis (Munsen et al., Analyt Biochem (1980) 107:220-39). Specific binding may be assessed using techniques known in the art including but not limited to competition ELISA, BIACORE® assays and/or KINEXA® assays. An antibody that preferentially or specifically binds (used interchangeably herein) to a target antigen or target epitope is a term well understood in the art, and methods to determine such specific or preferential binding are also known in the art. An antibody is said to exhibit specific or preferential binding if it reacts or associates more frequently, more rapidly, with greater duration and/or with greater affinity with a particular antigen or epitope than it does with alternative antigens or epitopes. An antibody specifically or preferentially binds to a target if it binds with greater affinity, avidity, more readily, and/or with greater duration than it binds to other substances. Also, an antibody specifically or preferentially binds to a target if it binds with greater affinity, avidity, more readily, and/or with greater duration to that target in a sample than it binds to other substances present in the sample. For example, an antibody that specifically or preferentially binds to a HER2 epitope is an antibody that binds this epitope with greater affinity, avidity, more readily, and/or with greater duration than it binds to other HER2 epitopes or non-HER2 epitopes. It is also understood by reading this definition, for example, that an antibody which specifically or preferentially binds to a first target antigen may or may not specifically or preferentially bind to a second target antigen. As such, specific binding and preferential binding do not necessarily require (although it can include) exclusive binding.

### Pharmaceutical Compositions

In some embodiments, the bispecific binding agents, nucleic acids, and recombinant cells of the disclosure can be incorporated into compositions, including pharmaceutical compositions. Such compositions typically include the bispecific binding agents, nucleic acids, and/or recombinant cells, and a pharmaceutically acceptable excipient, e.g., a carrier.

Bispecific binding agents of the disclosure can be administered using formulations used for administering antibodies and antibody-based therapeutics, or formulations based thereon. Nucleic acids of the disclosure are administered using formulations used for administering oligonucleotides, antisense RNA agents, and/or gene therapies such as CRISPR/Cas9 based therapeutics.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that it can be administered by syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the bispecific binding agents of the disclosure are administered by transfection or infection with nucleic acids encoding them, using methods known in the art, including but not limited to the methods described in McCaffrey et al., Nature (2002) 418:6893, Xia et al., Nature Biotechnol (2002) 20:1006-10, and Putnam, Am J Health Syst Pharm (1996) 53:151-60, erratum at Am J Health Syst Pharm (1996) 53:325.

Bispecific binding agents of the disclosure can be administered using a formulation comprising a fusogenic carrier. These are carriers capable of fusing with the plasma membrane of a mammalian cell. Fusogenic carriers include, without limitation, membrane-encapsulated viral particles and carriers based thereon, exosomes and microvesicles (see, e.g., Y. Yang et al., J Extracellular Vessicles (2018) 7:144131), fusogenic liposomes (see, e.g., Bailey et al., U.S. Pat. No. 5,552,155; Martin et al., U.S. Pat. No. 5,891,468; Holland et al., U.S. Pat. No. 5,885,613; and Leamon, U.S. Pat. No. 6,379,698).

### Methods of the Disclosure

The present disclosure provides, among others, a method of treating a disorder in a subject. The method includes administering to a subject in need thereof, a therapeutically effective amount of the bispecific binding agent, the nucleic acid, the vector, the engineered cell, or the pharmaceutical composition provided herein. The disorder that can be treated by the various compositions described herein can be a neoplastic disorder, an inflammatory disease, metabolic disorder, an endocrine disorder, and a neurological disorder.

In some embodiments, the condition to be treated includes a neoplastic disorder. Some non-limiting neoplastic disorders that can be treated by the various compositions described herein include, without being limited to, breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer.

In some embodiments, the condition to be treated includes an inflammatory disease. Some non-limiting inflammatory diseases that can be treated by the various compositions described herein include, without being limited to, inflammatory intestinal disease, rheumatoid arthritis, lupus, Crohn's disease, and ulcerative colitis.

In some embodiments, the condition to be treated includes a metabolic disorder. A metabolic disorder generally refers to a disorder that negatively alters the body's processing and distribution of macronutrients such as proteins, lipids, and carbohydrates. For example, metabolic disorders can happen when abnormal chemical reactions in the body alter the normal metabolic process. Metabolic disorders can also include inherited single gene anomalies, most of which are autosomal recessive. Further, metabolic disorders can be complications of severe diseases or conditions, including liver or respiratory failure, cancer, chronic obstructive pulmonary disease (COPD, includes emphysema and chronic bronchitis), and HIV/AIDS. Some non-limiting metabolic disorders that can be treated by the various compositions described herein include, without being limited to, diabetes, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick, phenylketonuria (PKU), Porphyria, Tay-Sachs disease, and Wilson's disease.

In some embodiments, the condition to be treated includes an endocrine disorder. Some non-limiting neurological disorders that can be treated by the various compositions described herein include, without diabetes mellitus, acromegaly (overproduction of growth hormone), Addison's disease (decreased production of hormones by the adrenal glands), Cushing's syndrome (high cortisol levels for extended periods of time), Graves' disease (type of hyperthyroidism resulting in excessive thyroid hormone production), Hashimoto's thyroiditis (autoimmune disease resulting in hypothyroidism and low production of thyroid hormone), hyperthyroidism (overactive thyroid), hypothyroidism (underactive thyroid), and prolactinoma (overproduction of prolactin by the pituitary gland).

In some embodiments, the condition to be treated includes a neurological disorder. Some non-limiting neurological disorders that can be treated by the various compositions described herein include, without being limited to, neurodegenerative disorders (e.g., Parkinson's, or Alzheimer's) or autoimmune disorders (e.g., multiple sclerosis) of the central nervous system; memory loss; long term and short term memory disorders; learning disorders; autism, depression, benign forgetfulness, childhood learning disorders, close head injury, and attention deficit disorder; autoimmune disorders of the brain, neuronal reaction to viral infection; brain damage; depression; psychiatric disorders such as bi-polarism, schizophrenia and the like; narcolepsy/sleep disorders (including circadian rhythm disorders, insomnia and narcolepsy); severance of nerves or nerve damage; severance of the cerebrospinal nerve cord (CNS) and any damage to brain or nerve cells; neurological deficits associated with AIDS; tics (e.g. Giles de la Tourette's syndrome); Huntington's chorea, schizophrenia, traumatic brain injury, tinnitus, neuralgia, especially trigeminal neuralgia, neuropathic pain, inappropriate neuronal activity resulting in neurodysthesias in diseases such as diabetes, MS and motor neurone disease, ataxias, muscular rigidity (spasticity) and temporomandibular joint dysfunction; Reward Deficiency Syndrome (RDS) behaviors in a subject. In some exemplary embodiments, the neurological disorders encompassed herein includes Parkinson's disease, Alzheimer's disease, and multiple sclerosis.

In some embodiments, the disclosure described herein provides for methods of degrading a target protein on a surface on a target cell. In some embodiments, the method of degrading a target protein on a surface on a target cell comprises degrading a target protein on a surface on a cancer cell. In some embodiments, the method of degrading a target protein on a surface on a target cell comprises degrading a target protein on a surface on a solid cancer cell. In some embodiments, the method of degrading a target protein on a surface on a target cell comprises degrading a target protein on a surface on a bladder cancer cell. In some embodiments the target protein is CDCP1. In some embodiments, the target cell expresses Nectin-4 and CDCP1. In some embodiments, the target cell expresses Nectin-4 and a normal, non-target cell does not express Nectin-4. In some embodiments, the target cell expresses Nectin-4 and a normal, non-target cell expresses levels of Nectin-4 that are equal to or lower than the target cell. In some embodiments, the target cell expresses CDCP1 and a normal, non-target cell does not express CDCP1. In some embodiments, the target cell expresses CDCP1 and a normal, non-target cell expresses levels of CDCP1 that are equal to or lower than the target cell.

In some embodiments, a bispecific binding agent comprising a first binding domain that specifically binds to Nectin-4 and a second binding domain that comprises a second binding domain that specifically binds to CDCP1 selectively targets a cancer cell. In some embodiments, a bispecific binding agent comprising a first binding domain that specifically binds to Nectin-4 and a second binding domain that comprises a second binding domain that specifically binds to CDCP1 selectively targets a solid cancer cell. In some embodiments, a bispecific binding agent comprising a first binding domain that specifically binds to Nectin-4 and a second binding domain that comprises a second binding domain that specifically binds to CDCP1 selectively targets a bladder cancer cell.

In some embodiments, a bispecific binding agent comprising a first binding domain that specifically binds to Nectin-4 and a second binding domain that comprises a second binding domain that specifically binds to CDCP1 selectively degrades CDCP1 on a target cell. In some embodiments, a bispecific binding agent comprising a first binding domain that specifically binds to Nectin-4 and a second binding domain that comprises a second binding domain that specifically binds to CDCP1 selectively degrades CDCP1 on a target cancer cell. In some embodiments, a bispecific binding agent comprising a first binding domain that specifically binds to Nectin-4 and a second binding domain that comprises a second binding domain that specifically binds to CDCP1 selectively degrades CDCP1 on a target solid cancer cell. In some embodiments, a bispecific binding agent comprising a first binding domain that specifically binds to Nectin-4 and a second binding domain that comprises a second binding domain that specifically binds to CDCP1 selectively degrades CDCP1 on a target bladder cancer cell.

In some embodiments, a method of selectively degrading CDCP1 on a target cell comprising contacting a bispecific binding agent comprising a first binding domain that specifically binds to Nectin-4 and a second binding domain that comprises a second binding domain that specifically binds to CDCP1. In some embodiments, a method of selectively degrading CDCP1 on a target cancer cell comprising contacting a bispecific binding agent comprising a first binding domain that specifically binds to Nectin-4 and a second binding domain that comprises a second binding domain that specifically binds to CDCP1. In some embodiments, a method of selectively degrading CDCP1 on a target solid cancer cell comprising contacting a bispecific binding agent comprising a first binding domain that specifically binds to Nectin-4 and a second binding domain that comprises a second binding domain that specifically binds to CDCP1. In some embodiments, a method of selectively degrading CDCP1 on a target bladder cancer cell comprising contacting a bispecific binding agent comprising a first binding domain that specifically binds to Nectin-4 and a second binding domain that comprises a second binding domain that specifically binds to CDCP1.

In some embodiments, the bispecific binding agent comprising a first binding domain which specifically binds to Nectin-4 and a second binding domain which specifically binds to CDCP1 contacts a target cancer cell. In some embodiments, the bispecific binding agent comprising a first binding domain which specifically binds to Nectin-4 and a second binding domain which specifically binds to CDCP1 contacts a target bladder cancer cell. In some embodiments, the bispecific binding agent comprising a first binding domain which specifically binds to Nectin-4 and a second binding domain which specifically binds to CDCP1 contacts a target cancer cell and decreases expression of CDCP1 on the cancer cell by at least 40%, at least 50%, at least 55%, at least 60%, at least 65%, at least 70%, at least 75%, or at least 80%. In some embodiments, the bispecific binding agent comprising a first binding domain which specifically binds to Nectin-4 and a second binding domain which specifically binds to CDCP1 contacts a target cancer cell and decreases expression of CDCP1 on the cancer cell by about 40%-80%, about 50%-80%, about 60%-80%, about 70%-80%, about 40%-70%, about 50%-70%, about 60%-70%, about 40%-60%, or about 50%-60%. In some embodiments, expression of CDCP1 on a target cell is determined relative to expression of CDCP1 on a control cancer cell not contacted with the binding agent.

### Administration of Bispecific Binding Agents

Administration of any one or more of the therapeutic compositions described herein, e.g., bispecific binding agents, nucleic acids, recombinant cells, and pharmaceutical compositions, can be used to treat individuals having a condition described herein. In some embodiments, the bispecific binding agents, nucleic acids, recombinant cells, and pharmaceutical compositions are incorporated into therapeutic compositions for use in methods down-regulating or inactivating T cells, such as CAR-T cells.

Accordingly, in one aspect, provided herein are methods for inhibiting an activity of a target cell in an individual, the methods comprising the step of administering to the individual a first therapy including one or more of the bispecific binding agents, nucleic acids, recombinant cells, and pharmaceutical compositions provided herein, wherein the first therapy inhibits an activity of the target cell by degrading a target surface protein. For example, an activity of the target cell may be inhibited if its proliferation is reduced, if its pathologic or pathogenic behavior is reduced, if it is destroyed or killed, or the like. Inhibition includes a reduction of the measured quantity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits the target cell in the individual by expression of bispecific binding agents. Generally, the target cell of the disclosed methods can be any cell such as, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a bladder cancer cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

Bispecific binding agents of the disclosure are typically administered in solution or suspension formulation by injection or infusion. In an embodiment, a bispecific binding agent is administered by injection directly into a tumor mass. In another embodiment, a bispecific binding agent is administered by systemic infusion.

The effective dose of the bispecific binding agents can be determined by a skilled person in the field, e.g. a physician. The effective dose of any given bispecific binding agent may depend on the binding affinity for each of the ligands, and the degree of expression of each of the ligands. The range of effective concentrations, however, can be determined by one of ordinary skill in the art, using the disclosure and the experimental protocols provided herein. Similarly, using the effective concentration one can determine the effective dose or range of dosages required for administration.

Depending on the disease or disorder to be treated, the severity and extent of the disease, the subject's health, and the co-administration of other therapies, repeated doses may be administered. Alternatively, a continuous administration may be required. It is expected, however, that the bispecific binding agent will remain in proximity to the cell so that each molecule of bispecific binding agent can ubiquitinate and degrade multiple molecules of target surface protein. Thus, the bispecific binding agents of the disclosure may require lower doses, or less frequent administration, than therapies based on antibody competitive binding.

In some embodiments, a method for treating cancer in a subject comprises administering to a subject a binding agent, wherein the binding agent comprises a first binding domain that specifically binds to an endogenous internalizing receptor, wherein the endogenous internalizing receptor is expressed on a target cell, wherein the endogenous internalizing receptor comprises Nectin-4, and a second binding domain that specifically binds to the target protein, wherein the target protein comprises CDCP1. In some embodiments, the method for treating cancer in a subject comprises administering to a subject a binding agent, wherein the binding agent comprises a first binding domain that specifically binds to an endogenous internalizing receptor, wherein the endogenous internalizing receptor is expressed on a target cell, wherein the endogenous internalizing receptor comprises Nectin-4, and a second binding domain that specifically binds to the target protein, wherein the target protein comprises CDCP1, and the method results in a decrease in CDCP1 expression on the target cell.

In some embodiments, the method of treating cancer comprises a decrease in expression of the target protein on the target cell. In some embodiments, the method of treating cancer comprises a decrease in expression of CDCP1 on the target cell. In some embodiments, the method of treating cancer comprises administration of the bispecific binding agent as an individual therapeutic. In some embodiments, the method of treating cancer comprises administration of the bispecific binding agent as a combination therapeutic. In some embodiments, the combination therapeutic comprises administering the bispecific binding agent before, after, or at the same time as an additional therapeutic. In some embodiments, the additional therapeutic comprises a standard of care treatment. In some embodiments, nonlimiting examples of standard of care treatments comprise cytotoxic agents, immunotherapies, radiation, chemotherapies, surgery, hormone therapies, or a combination thereof.

### Administration of Recombinant Cells to an Individual

In some embodiments, the methods involve administering the recombinant cells to an individual who is in need of such method. This administering step can be accomplished using any method of implantation known in the art. For example, the recombinant cells can be injected directly into the individual's bloodstream by intravenous infusion or otherwise administered to the individual.

The terms “administering”, “introducing”, and “transplanting” are used interchangeably herein to refer to methods of delivering recombinant cells expressing the bispecific binding agents provided herein to an individual. In some embodiments, the methods comprise administering recombinant cells to an individual by a method or route of administration that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even long-term engraftment for the lifetime of the individual.

When provided prophylactically, in some embodiments, the recombinant cells described herein are administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source (i.e., the human subject being treated). In some embodiments, the recombinant cells are expanded in culture prior to administration to an individual in need thereof.

In some embodiments, the delivery of a composition comprising recombinant cells (i.e., a composition comprising a plurality of recombinant cells a bispecific binding agent provided herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. Injection modes include, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment with a composition for the treatment of a disease or condition can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting disease progression, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments, the individual is a mammal. In some embodiments, the mammal is human. In some embodiments, the individual has or is suspected of having a disease associated with cell signaling mediated by a cell surface protein (e.g., a membrane-associated target protein) or a soluble target protein. In some embodiments, the disorder is a neoplastic disorder, an inflammatory disease, and a neurological disorder.

### Systems and Kits

Also provided herein are systems and kits including the bispecific binding agents, recombinant nucleic acids, recombinant cells, or pharmaceutical compositions provided and described herein as well as written instructions for making and using the same. For example, provided herein, in some embodiments, are systems and/or kits that include one or more of a bispecific binding agent as described herein, a recombinant nucleic acid as described herein, a recombinant cell as described herein, or a pharmaceutical composition as described herein. In some embodiments, the systems and/or kits of the disclosure further include one or more syringes (including pre-filled syringes) and/or catheters used to administer one any of the provided bispecific binding agents, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the bispecific binding agents.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### Example 1: Methods

Cell lines. Cell lines were grown and maintained in T75 (Thermo Fisher Scientific) flasks at 37° C. and 5% CO₂. HT-1376 cells were grown in Eagle's Minimum Essential Medium (EMEM) supplemented with 10% fetal bovine serum (FBS) and 1% penicillin/streptomycin.

Bispecific antibody expression. Half IgGs were expressed and purified from Expi293 BirA cells using transient transfection (Expifectamine, Thermo Fisher Scientific). Enhancers were added 20 hrs after transfection. Cells were incubated for 5 days at 37° C. and 8% C02. Media was then harvested by centrifugation at 4,000×g for 20 min. Knob half IgGs were purified by Ni-NTA affinity chromatography and buffer exchanged into PBS containing 20% glycerol and concentrated. Hole half IgGs were purified by Protein A affinity chromatography, buffer exchanged into PBS containing 20% glycerol, and concentrated. Knob and hole half IgGs were then recombined under reducing conditions (10 mM Tris pH 7.5, 100 mM NaCl, 20% 800 mM L-Arg pH 10 plus 200-fold excess reduced glutathione). Fully formed bispecifics were then purified by Ni-NTA affinity chromatography, buffer exchanged into PBS containing 20% glycerol, concentrated, and flash frozen for storage at −80° C. Purity and integrity of all proteins were assessed by SDS-PAGE.

Degradation experiments. Cells were plated in 6- or 12-well plates and grown to ˜70% confluency before treatment. Media was aspirated and cells were treated with bispecifics or control antibodies in complete growth medium. For soluble ligand uptake experiments, biotinylated soluble ligand was pre-incubated with streptavidin-647 at 37° C. for 30 min, then mixed with bispecific or control antibodies and added to cells. After incubation at 37° C. for the designated time point, cells were washed with phosphate-buffered saline (PBS), lifted with versene, and harvested by centrifugation at 300×g for 5 min at 4° C. Samples were then tested by western blotting or flow cytometry to quantify protein levels.

Western blotting: Cell pellets were lysed with 1×RIPA buffer containing cOmplete mini protease inhibitor cocktail (Sigma-Aldrich) at 4° C. for 40 min. Lysates were centrifuged at 16,000×g for 10 min at 4° C. and protein concentrations were normalized using BCA assay (Pierce). 4×NuPAGE LDS sample buffer (Invitrogen) and 2-mercaptoethanol (BME) was added to the lysates and boiled for 10 min. Equal amounts of lysates were loaded onto a 4-12% Bis-Tris gel and ran at 200V for 37 min. The gel was incubated in 20% ethanol for 10 min and transferred onto a polyvinylidene difluoride (PVDF) membrane. The membrane was blocked in PBS with 0.1% Tween-20+5% bovine serum albumin (BSA) for 30 min at room temperature with gentle shaking. Membranes were incubated overnight with primary antibodies at respective dilutions at 4° C. with gentle shaking in PBS+0.2% Tween-20+5% BSA. Membranes were washed four times with tris-buffered saline (TBS)+0.1% Tween-20 and then co-incubated with HRP-anti-rabbit IgG (Cell Signaling Technologies, 7074A, 1:2000) and 680RD goat anti-mouse IgG (LI-COR, 926-68070, 1:10000) in PBS+0.2% Tween-20+5% BSA for 1 hr at room temperature. Membranes were washed four times with TBS+0.1% Tween-20, then washed with PBS. Membranes were imaged using an OdysseyCLxImager (LI-COR). SuperSignal West Pico PLUS Chemiluminescent Substrate (Thermo Fisher Scientific) was then added and imaged using a ChemiDoc Imager (BioRad). Band intensities were quantified using Image Studio Software (LI-COR).

### Example 2: Internalization Platform can Target CDCP1

This Example sought to determine whether the internalization platform could be applied towards the degradation of therapeutically relevant cell surface proteins.

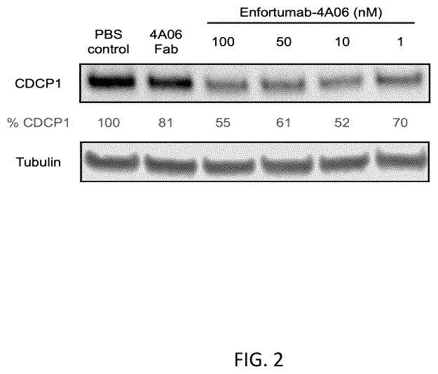

First, CDCP1 was targeted, which is frequently upregulated in cancer. CDCP1 had been previously identified to be upregulated on the surface of KRAS^{G12V} transformed cells. Enfortumab-4A06 antibody, which binds CDCP1 and Nectin-4, was used as the internalization construct. Next, HT-1376 cells known to express CDCP1 and Nectin-4 were treated with Enfortumab-4A06 antibody and observed significant degradation of CDCP1 after 24 hrs (FIG. 2).

### Example 3: Exemplary Internalization Platform Constructs

Some constructs shown below in Table 5 have been generated and successfully expressed. The remainder will be completed. All of these constructs will be tested using protocols described in Example 1.

TABLE 5

| | | SEQ ID |
|---|---|---|
| Protein Name | Sequence | NO: |
| Enfortumab- | Knob HC: | 57 |
| 4A06 | EVQLVESGGGLVQPGGSLRLSCAASGFTFSSYNMNWVR | |
| | QAPGKGLEWVSYISSSSSTIYYADSVKGRFTISRDNAKNS | |
| | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | 58 |
| | DIQMTQSPSSVSASVGDRVTITCRASQGISGWLAWYQQK | |
| | PGKAPKFLIYAASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 59 |
| | EISEVQLVESGGGLVQPGGSLRLSCAASGFNLSYYYIHW | |
| | VRQAPGKGLEWVASIYSSSSYTSYADSVKGRFTISADTS | |
| | KNTAYLQMNSLRAEDTAVYYCARAYYGFDYWGQGTL | |
| | VTVSSASTKGPSVFPLAPSSKSTSGGTAALGCLVKDYFPE | |
| | PVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLT | |
| | VLHQDWLNGKEYKCKVSNKALPAPIEKTISKAKGQPREP | |
| | QVYTLPPIRELMTSNQVSLSCAVKGFYPSDIAVEWESNG | |
| | QPENNYKTTPPVLDSDGSFFLVSKLTVDKSRWQQGNVF | |
| | SCSVMHEALHNHYTQKSLSLSPGK | |
| | Hole LC: | 60 |
| | DIQMTQSPSSLSASVGDRVTITCRASQSVSSAVAWYQQK | |
| | PGKAPKLLIYSASSLYSGVPSRFSGSRSGTDFTLTISSLQP | |
| | EDFATYYCQQSYYYYPITFGQGTKVEIKRTVAAPSVFIFP | |
| | PSDSQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGECGGSDYKDDDDK | |
| Enfortumab- | Knob HC: | 57 |
| Tecentriq | EVQLVESGGGLVQPGGSLRLSCAASGFTFSSYNMNWVR | |
| | QAPGKGLEWVSYISSSSSTIYYADSVKGRFTISRDNAKNS | |
| | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | 58 |
| | DIQMTQSPSSVSASVGDRVTITCRASQGISGWLAWYQQK | |
| | PGKAPKFLIYAASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 63 |
| | EVQLVESGGGLVQPGGSLRLSCAASGFTFSDSWIHWVR | |
| | QAPGKGLEWVAWISPYGGSTYYADSVKGRFTISADTSK | |
| | NTAYLQMNSLRAEDTAVYYCARRHWPGGFDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPS | |
| | SSLGTQTYICNVNHKPSNTKVDKKVEPKSCEPKSCDKTH | |
| | TCPPCPAPELLGGPSVFLFPPKPKDTLMISRTPEVTCVVV | |
| | DVSHEDPEVKFNWYVDGVEVHNAKTKPREEQYGSTYR | |
| | VVSVLTVLHQDWLNGKEYKCKVSNKALPAPIEKTISKA | |
| | KGQPREPQVYTLPPSRDELTKNQVSLSCAVKGFYPSDIA | |
| | VEWESNGQPENNYKTTPPVLDSDGSFFLVSKLTVDKSR | |
| | WQQGNVFSCSVMHEALHNHYTQKSLSLSPGKGGSGAW | |
| | SHPQFEK | |
| | Hole LC: | 64 |
| | DIQMTQSPSSLSASVGDRVTITCRASQDVSTAVAWYQQK | |
| | PGKAPKLLIYSASFLYSGVPSRFSGSGSGTDFTLTISSLQP | |
| | EDFATYYCQQYLYHPATFGQGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| Enfortumab- | Knob HC: | 57 |
| Trastuzumab | EVQLVESGGGLVQPGGSLRLSCAASGFTFSSYNMNWVR | |
| | QAPGKGLEWVSYISSSSSTIYYADSVKGRFTISRDNAKNS | |
| | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | 58 |
| | DIQMTQSPSSVSASVGDRVTITCRASQGISGWLAWYQQK | |
| | PGKAPKFLIYAASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 67 |
| | EVQLVESGGGLVQPGGSLRLSCAASGFNIKDTYIHWVRQ | |
| | APGKGLEWVARIYPTNGYTRYADSVKGRFTISADTSKNT | |
| | AYLQMNSLRAEDTAVYYCSRWGGDGFYAMDYWGQGT | |
| | LVTVSSASTKGPSVFPLAPSSKSTSGGTAALGCLVKDYFP | |
| | EPVTVSWNSGALTSGVHTFPAVLQSSGLYSLSSVVTVPS | |
| | SSLGTQTYICNVNHKPSNTKVDKKVEPKSCDKTHTCPPC | |
| | PAPELLGGPSVFLFPPKPKDTLMISRTPEVTCVVVDVSHE | |
| | DPEVKFNWYVDGVEVHNAKTKPREEQYGSTYRVVSVL | |
| | TVLHQDWLNGKEYKCKVSNKALPAPIEKTISKAKGQPR | |
| | EPQVYTLPPSRDELTKNQVSLSCAVKGFYPSDIAVEWES | |
| | NGQPENNYKTTPPVLDSDGSFFLVSKLTVDKSRWQQGN | |
| | VFSCSVMHEALHNHYTQKSLSLSPGKGGSGAWSHPQFE | |
| | K | |
| Enfortumab- | Hole LC: | 68 |
| Cetuximab | DIQMTQSPSSLSASVGDRVTITCRASQDVNTAVAWYQQ | |
| | KPGKAPKLLIYSASFLYSGVPSRFSGSRSGTDFTLTISSLQ | |
| | PEDFATYYCQQHYTTPPTFGQGTKLEIKRTVAAPSVFIFP | |
| | PSDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Knob HC: | 57 |
| | EVQLVESGGGLVQPGGSLRLSCAASGFTFSSYNMNWVR | |
| | QAPGKGLEWVSYISSSSSTIYYADSVKGRFTISRDNAKNS | |
| | LSLQMNSLRDEDTAVYYCARAYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKRVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSRDELTKNQVSLWCLVKGFYPSDIAVEWESNG | |
| | QPENNYKTTPPVLDSDGSFFLYSKLTVDKSRWQQGNVF | |
| | SCSVMHEALHNHYTQKSLSLSPGKGGSHHHHHH | |
| | Knob LC: | 58 |
| | DIQMTQSPSSVSASVGDRVTITCRASQGISGWLAWYQQK | |
| | PGKAPKFLIYAASTLQSGVPSRFSGSGSGTDFTLTISSLQP | |
| | EDFATYYCQQANSFPPTFGGGTKVEIKRTVAAPSVFIFPP | |
| | SDEQLKSGTASVVCLLNNFYPREAKVQWKVDNALQSG | |
| | NSQESVTEQDSKDSTYSLSSTLTLSKADYEKHKVYACEV | |
| | THQGLSSPVTKSFNRGEC | |
| | Hole HC: | 71 |
| | QVQLKQSGPGLVQPSQSLSITCTVSGFSLTNYGVHWVRQ | |
| | SPGKGLEWLGVIWSGGNTDYNTPFTSRLSINKDNSKSQV | |
| | FFKMNSLQSNDTAIYYCARALTYYDYEFAYWGQGTLVT | |
| | VSAASTKGPSVFPLAPSSKSTSGGTAALGCLVKDYFPEP | |
| | VTVSWNSGALTSGVHTFPAVLQSSGLYSLSSVVTVPSSS | |
| | LGTQTYICNVNHKPSNTKVDKKVEPKSCDKTHTCPPCPA | |
| | PELLGGPSVFLFPPKPKDTLMISRTPEVTCVVVDVSHEDP | |
| | EVKFNWYVDGVEVHNAKTKPREEQYNSTYRVVSVLTV | |
| | LHQDWLNGKEYKCKVSNKALPAPIEKTISKAKGQPREPQ | |
| | VYTLPPSRDELTKNQVSLSCAVKGFYPSDIAVEWESNGQ | |
| | PENNYKTTPPVLDSDGSFFLVSKLTVDKSRWQQGNVFSC | |
| | SVMHEALHNHYTQKSLSLSPGK | |
| | Hole LC: | 72 |
| | DILLTQSPVILSVSPGERVSFSCRASQSIGTNIHWYQQRTN | |
| | GSPRLLIKYASESISGIPSRFSGSGSGTDFTLSINSVESEDIA | |
| | DYYCQQNNNWPTTFGAGTKLELKRTVAAPSVFIFPPSDE | |
| | QLKSGTASVVCLLNNFYPREAKVQWKVDNALQSGNSQ | |
| | ESVTEQDSKDSTYSLSSTLTLSKADYEKHKVYACEVTHQ | |
| | GLSSPVTKSFNRGEC | |

TABLE 6
Cloned sequences:

| Protein | | SEQ ID |
|---|---|---|
| Name | Sequence | NO |
| Sacituzumab | HC: | 73 |
| knob | QVQLQQSGSELKKPGASVKVSCKASGYTFTNYGMNWVK | |
| | QAPGQGLKWMGWINTYTGEPTYTDDFKGRFAFSLDTSVS | |
| | TAYLQISSLKADDTAVYFCARGGFGSSYWYFDVWGQGSL | |
| | VTVSSASTKGPSVFPLAPSSKSTSGGTAALGCLVKDYFPEP | |
| | VTVSWNSGALTSGVHTFPAVLQSSGLYSLSSVVTVPSSSL | |
| | GTQTYICNVNHKPSNTKVDKRVEPKSCDKTHTCPPCPAPE | |
| | LLGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTLP | |
| | PSRDELTKNQVSLWCLVKGFYPSDIAVEWESNGQPENNY | |
| | KTTPPVLDSDGSFFLYSKLTVDKSRWQQGNVFSCSVMHE | |
| | ALHNHYTQKSLSLSPGKGGSHHHHHH | |
| | LC: | 74 |
| | DIQLTQSPSSLSASVGDRVSITCKASQDVSIAVAWYQQKPG | |
| | KAPKLLIYSASYRYTGVPDRFSGSGSGTDFTLTISSLQPEDF | |
| | AVYYCQQHYITPLTFGAGTKVEIKRTVAAPSVFIFPPSDEQ | |
| | LKSGTASVVCLLNNFYPREAKVQWKVDNALQSGNSQESV | |
| | TEQDSKDSTYSLSSTLTLSKADYEKHKVYACEVTHQGLSS | |
| | PVTKSFNRGEC | |

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

1-42. (canceled)

43. A method of degrading a target protein on a surface of a target cell, the method comprising:
contacting an endogenous internalizing receptor and the target protein on the surface of the target cell with a binding agent, wherein the binding agent comprises:
(i) a first binding domain that specifically binds to an endogenous internalizing receptor, wherein the endogenous internalizing receptor comprises Nectin-4;
(ii) a second binding domain that specifically binds to the target protein, wherein the target protein comprises CDCP1.

44. The method of claim 43, wherein following the contacting, the target protein is internalized with the endogenous internalizing receptor into the target cell and the target protein is degraded.

45. The method of claim 43, wherein the binding agent is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

46. The method of claim 45, wherein the first binding domain binds to an epitope of the endogenous internalizing receptor on the target cell, wherein the epitope comprises at least 80% sequence identity to an epitope to which Enfortumab binds.

47-49. (canceled)

50. The method of claim 49, wherein the first binding domain comprises a first binding domain variable heavy chain, and wherein the first binding domain variable heavy chain comprises at least 80%, sequence identity to SEQ ID NO: 57.

51-52. (canceled)

53. The method of claim 45, wherein the first binding domain comprises a first binding domain variable light chain, and wherein the first binding domain variable light chain comprises at least 80% sequence identity to SEQ ID NO: 58.

54-56. (canceled)

57. The method of claim 43, wherein the second binding domain comprises a second binding domain variable heavy chain, and wherein the second binding domain variable heavy chain comprises at least 80% sequence identity to SEQ ID NO: 59.

58-59. (canceled)

60. The method of claim 45, wherein the second binding domain comprises a second binding domain variable light chain, and wherein the second binding domain variable light chain comprises at least 80% sequence identity to SEQ ID NO: 60.

61-62. (canceled)

63. The method of claim 44, wherein the endogenous internalizing receptor is recycled to the target cell surface following the internalization of the binding agent.

64. The method of claim 43, wherein the endogenous internalizing receptor is degraded.

65. The method of claim 43, wherein the target cell is a cancer cell.

66. The method of claim 65, wherein the cancer cell is from a solid tumor, e.g., bladder cancer.

67. The method of claim 66, wherein expression of CDCP1 on the cancer cell decreases following contact with the bispecific binding agent, as compared to a control cancer cell that is not contacted with the binding agent.

68. (canceled)

69. The method of claim 43, wherein the method increases the susceptibility of the cancer cell to cancer therapeutic agents.

70. The method of claim 69, wherein the cancer therapeutic agent is a cytotoxic agent.

71. The method of claim 65, wherein the method reduces proliferation of the cancer cell.

72. The method of claim 65, wherein the method increases death of the cancer cell.

73. The method of claim 1, wherein the contacting is performed in vivo.

74. A method for treating cancer in a subject, the method comprising:
administering to a subject a binding agent, wherein the binding agent comprises:
(i) a first binding domain that specifically binds to an endogenous internalizing receptor, wherein the endogenous internalizing receptor is expressed on a target cell, wherein the endogenous internalizing receptor comprises Nectin-4;
(ii) a second binding domain that specifically binds to the target protein, wherein the target protein comprises CDCP1.

75. The method of claim 74, wherein the cancer is breast cancer, B cell lymphoma, pancreatic cancer, Hodgkin's lymphoma, ovarian cancer, prostate cancer, mesothelioma, lung cancer, non-Hodgkin's B-cell (B-NHL) lymphoma, melanoma, chronic lymphocytic leukemia, acute lymphocytic leukemia, neuroblastoma, glioma, glioblastoma, bladder cancer, and colorectal cancer.

76. The method of claim 75, wherein the cancer is bladder cancer.

77. A bispecific binding agent comprising:
(a) a first binding domain that specifically binds to Nectin-4, wherein Nectin-4 is associated with a membrane of a target cell; and
(b) a second binding domain that specifically binds to a target protein, wherein the target protein is selected from the group consisting of CDCP1, PD-L1, HER2, and EGFR.

78. The bispecific binding agent of claim 77, wherein the bispecific binding agent is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

79. The bispecific binding agent of claim 78, wherein the first binding domain binds to an epitope of the endogenous internalizing receptor on the target cell, wherein the epitope comprises at least 80% sequence identity to an epitope to which Enfortumab binds.

80-82. (canceled)

83. The bispecific binding agent of claim 82, wherein the first binding domain comprises a first binding domain variable heavy chain, and wherein the first binding domain variable heavy chain comprises at least 80% sequence identity to SEQ ID NO: 57.

84-85. (canceled)

86. The bispecific binding agent of claim 77, wherein the first binding domain comprises a first binding domain variable light chain, and wherein the first binding domain variable light chain comprises at least 80% sequence identity to SEQ ID NO: 58.

87-89. (canceled)

90. The bispecific binding agent of claim 77, wherein the second binding domain comprises a second binding domain variable heavy chain, and wherein the second binding domain variable heavy chain comprises at least 80% sequence identity to SEQ ID NO: 59, 63, 67, or 71.

91-92. (canceled)

93. The bispecific binding agent of claim 77, wherein the second binding domain comprises a second binding domain variable light chain, and wherein the second binding domain variable light chain comprises at least 80% sequence identity to any one of SEQ ID NOs: 60, 64, 68, or 72.

94-95. (canceled)
